(19) United States
(12) Patent Application Publication
(10) Pub. No.: US 2014/0171795 A1
(43) Pub. Date: Jun. 19, 2014

(54) METHOD, APPARATUS AND SYSTEM FOR THE PERFORMANCE OF VALSALVA MANEUVERS

(71) Applicant: Cardiox Corporation, Columbus, OH (US)

(72) Inventors: Philip E. Eggers, Dublin, OH (US); Andrew R. Eggers, Ostrander, OH (US); Mark A. Mayerchak, Bothell, WA (US)

(73) Assignee: CARDIOX CORPORATION, Columbus, OH (US)

(21) Appl. No.: 14/018,295

(22) Filed: Sep. 4, 2013

Related U.S. Application Data
(60) Provisional application No. 61/696,409, filed on Sep. 4, 2012.

(51) Int. Cl. A61B 5/00; A61M 5/00; A61M 5/178; A61B 5/02

(52) U.S. Cl. CPC A61B 5/4884; A61B 5/02028; A61B 5/742; A61M 5/007; A61M 5/178
USPC 600/432; 600/508

(57) ABSTRACT

Methods, apparatus and systems for precisely controlling a Valsalva maneuver and the timing thereof in conjunction with testing a patient for a cardiac shunt, and for ensuring that a patient performing a Valsalva maneuver creates a required pressure using their diaphragm. Embodiments of a Valsalva maneuver mouthpiece assembly are also disclosed for use with the invention methods, apparatus and systems. The mouthpiece assembly is adapted to selectively operate between a state that produces resistance to the exhalation pressure of a patient into the mouthpiece and a state wherein the pressure resistance is rapidly removed so as to cause an involuntary exhalation of the air in the patient's lungs at a time of desired Valsalva maneuver release.

### CROSS-REFERENCE TO RELATED APPLICATIONS

This application claims the benefit of U.S. Provisional Application No. 61/696,409 filed on Sep. 4, 2012.

### BACKGROUND

The present invention generally relates to a method, system and apparatus for the performance of a maneuver essential for the detection of circulatory anomalies in the mammalian body. Important types of such anomalies involve the heart and include anomalies generally referred to as right-to-left cardiac shunts.

An anomaly commonly encountered in humans is an opening between the chambers of the heart, particularly an opening between the left and right atria (i.e., an Atrial Septal Defect (ASD) that creates a right-to-left atrial shunt), or between the left and right ventricles (i.e., a Ventricular Septal Defect (VSD) that creates a right-to-left ventricular shunt. A right-to-left shunt may occur as a defect within the vasculature leading to and from the heart, for example a Pulmonary Arteriovenous Malformation (PAVM) may be present, reflecting a direct connection between the pulmonary vein and pulmonary artery. Alternatively, a right-to-left shunt may occur as a defect between great vessels. For example, a Patent Ductus Arteriosus may be present, allowing shunting between the aortic arch and the pulmonary artery.

The passage of a thrombotic embolism via a cardiac right-to-left shunt is a widely recognized cause of cerebral ischemia (e.g., stroke). Over 780,000 patients suffer strokes each year in the U.S. resulting in 250,000 stroke related deaths. The total cost associated with stroke was reported to be $66 billion in the U.S. in 2007. (Rosamond 2008). Of the patient population presenting with stroke or the early warning sign known as transient ischemic attack (TIA or mini stroke), as many as 260,000 are reported to be the result of a right-to-left shunt in the heart and/or pulmonary vasculature, allowing paradoxical emboli.

The most common form of right-to-left shunt is a patent foramen ovale (PFO), which is an opening in the wall of the heart that separates the right side of the heart from the left side of the heart. The right side of the heart receives oxygen-depleted blood from the body and then pumps this blood into the lungs for oxygenation. The lungs not only oxygenate the blood, but also serve as a “filter” for any blood clots or other emboli, and also metabolize other agents that naturally reside within the venous blood. During the fetal stage of development, an opening naturally exists between the right and left atria of the heart to enable circulation of the mother's oxygenated blood throughout the vasculature of the fetus. This opening between the right and left side of the fetal heart (known as the foramen ovale) permanently seals shut in consequence of the closure of an overlying tissue flap in about 80% of the population within the first eighteen months following birth. The noted flap often remains in a sealing orientation because of a higher pressure at the left side of the heart. However, in the remaining approximate 20% of the population, this opening fails to permanently close and is referred to as a patent foramen ovale or PFO.

Most of the population with a PFO never experience any symptoms or complications associated with the presence of a PFO, since many such PFOs are small enough to remain effectively “closed”, or emboli may not form and travel to the right atrium, or they may not pass through a PFO even if it is present and open; thus the paradoxical nature of these emboli. However, for more than 20% of the adult population, this normally closed flap covering the foramen ovale temporarily opens during various types of exertion or coughing, allowing blood to temporarily flow directly from the right side to the left side of the heart.

As a consequence, emboli such as blood clots or other active agents escaping through the PFO bypass the critical filtering functions of the lungs and flow through the temporarily open foramen ovale and directly to the left side of the heart. Once in the left side of the heart, these emboli pass directly into the arterial circulatory system. Since a significant portion of the blood exiting the left side of the heart flows to the brain, any unfiltered blood clots or agents, such as serotonin, may be delivered to the brain. The presence of these now cerebral emboli in the brain arterial flow can produce debilitating and life-threatening consequences. These consequences are known to include stroke, heart attack and are also now believed to be one of the causes of certain forms of severe migraine headaches. For further background on circulatory anomalies, see:
1) Banas, J., et al. American Journal of Cardiology 28: 467-471 (October 1971);
2) Castillo, C., et al. American Journal of Cardiology 17: 691-694 (May 1966);
3) Schwedt, T. J., et al., “Patent Foramen Ovale Migraine—Bringing Closure to the Subject.” Headache 46(4): 663-671 (2006);
4) Spies, C., et al., “Transcatheter Closure of Patent Foramen Ovale in Patients with Migraine Headache.” Journal of Interventional Cardiology 19(6): 552-557 (2006).

Transesophageal Echocardiography (TEE), involving an ultrasound transducer positioned in the patient's esophagus in close proximity to the heart, is widely used as part of the diagnostic evaluation of patients with cerebral ischemia. Numerous studies have demonstrated the value of TEE for the detection of a PFO or an ASD as a possible cause of cerebral ischemia. Currently, TEE, enhanced by an injected echo-contrast agent (e.g., a 10 ml solution containing contrast air bubbles), is used somewhat as a last resort. While the so-called TEE “bubble study” has not been reviewed by the U.S. Food and Drug Administration, and so is performed “off-label”, it is still is considered the “gold standard” for the detection of a cardiac right-to-left shunt. The air bubbles contained in the echo-contrast agent used for this test are essentially unable to pass through the pulmonary capillary bed. The echogenic air bubbles passing through a right-to-left shunt and entering the left atrium, within about three heart beats after said contrast arrives at the right atrium, produce visible images on the ultrasound monitor screen and ultrasound recording and indicate the presence and relative conductance of the right-to-left shunt based on the number of air bubbles observed in the left atrium. For further background on TEE methods, see for example:
5) O'Brien P J, Thiemann D R, McNamara R L, Roberts J W, Raska K, Oppenheimer S M, Lima J A C. “Usefulness of transeophageal echocardiography in predicting mortality and morbidity in stroke patients without clinically known cardiac sources of embolus.” American Journal of Cardiology 81: 1144-1151 (1998);
6) Leung D Y, Black I W, Cranney G B, Walsh W F, Grimm R A, Stewart W J, Thomas J D. “Selection of patients for Transesophageal Echocardiography after stroke and systemic embolic events.” Stroke 26: 1820-1824 (1995).
7) Rauh G, Fischereder M, Spengel F A. “Transesophageal Echocardiography in patients with focal cerebral ischemia of unknown cause.” Stroke 27: 691-694 (1996).
8) Nighoghossian N, Perinetti M, Barthelett M, Adeleine P, Trouillas P. “Potential cardioembolic sources of stroke in patients less than 60 years of age.” European Heart Journal 17: 590-594 (1996).

Alternatively, a test referred to as transthoracic echocardiography (TTE), which also uses an injected echo-contrast agent (containing air bubbles), can be used for the detection of a PFO or a ASD as a possible cause of cerebral ischemia. The air bubbles contained in the echo-contrast agent used for this test are essentially unable to pass through the pulmonary capillary bed. The echogenic air bubbles passing through a right-to-left shunt and entering the left atrium, within about three heart beats after said contrast arrives at the right atrium, produce visible images on the ultrasound monitor screen and indicate the presence and relative conductance of the right-to-left shunt based on the number of air bubbles observed in the left atrium. Unlike TEE, which requires insertion of an ultrasound transducer into the esophagus, TTE is performed by placing the ultrasound transducer on the surface of a patient's chest near the heart. For further background on the TTE methods, see for example:
9) Gonzalez-Alujas, T. et. al. “Diagnosis and Quantification of Patent Foramen Ovale. Which Is the Reference Technique? Simultaneous Study With Transcranial Doppler, Transthoracic and Transesophageal Echocardiography.” Rev. Esp. Cardiology 64(2): 133-139 (2011).

In addition to TEE and TTE, a cardiac right-to-left shunt can also be identified by the use of contrast-enhanced Transcranial Doppler (TCD) sonography. This technique is based on the detection of an intravenously injected contrast agent (containing air bubbles) within intracranial arteries, e.g., the middle cerebral arteries (MCAs). The air bubbles contained in the echo-contrast agent used for this test are essentially unable to pass through the pulmonary capillary bed. In case of a right-to-left shunt, the contrast agent bypasses the pulmonary capillary bed and enters the arterial circulation via a right-to-left shunt. The echogenic air bubbles passing through a right-to-left shunt and, upon entering the arterial circulation via the left atrium, produce microembolic signals (MES) during the TCD ultrasound recording, thus mimicking the pathway of paradoxical cerebral emboli. For further background on TCD methods, see for example:
10) Teague S M, Sharma M K. “Detection of paradoxical cerebral echo contrast embolization by Transcranial Doppler ultrasound.” Stroke 22: 740-745 (1991).
11) Ringelstein E B, Droste D W, Babikian V L, Evans D H, Grosset D G, Kaps M, Markus H S, Russell D, Siebler M. “Consensus on microembolus detection by Transcranial Doppler ultrasound.” Stroke 29: 725-729 (1998).

The contrast agent most widely used in the performance of TEE, TTE and TCD is agitated saline containing tiny air bubbles. The mean microbubble size for a 10% air-10% blood-80% saline mixture is 26.7±7.2 microns and for a 10% air-10% plasma-80% saline mixture is 25.3±7.4 microns. However, it is possible for some bubbles at the very small end of the size range to pass through the pulmonary capillary bed. For that reason, the timing or window for the observation of the presence of air bubbles in either the left atrium or middle cerebral arteries, relative to the performance of a maneuver such as the Valsalva maneuver, is critical following the injection of air bubble contrast agent.

Yet a fourth method for the detection of right-to-left cardiac shunts employs an injectable dye rather than air bubbles to detect the presence of a right-to-left cardiac shunt. A description of this method, apparatus and system for the detection of circulatory anomalies is described in co-pending U.S. patent application Ser. No. 12/754,888 filed Apr. 6, 2010 and Ser. No. 12/418,866 filed Apr. 6, 2009; in U.S. Provisional Application Nos. 61/156,723 filed Mar. 2, 2009 and 61/080,724 filed Jul. 15, 2008; and in PCT applications PCT/US09/50630 filed Jul. 15, 2009 and PCT/US11/31433 filed Apr. 6, 2011.

The presence of a right-to-left shunt is determined with this fourth method, apparatus and system by first deriving the magnitude of the peak amplitude of a measured indocyanine green (ICG) dye concentration for a premature shunt curve or inflection that may occur in advance of a normal indicator-dilution curve associated with ICG dye following a normal pathway through the lungs. A premature shunt curve or inflection can only occur if the ICG dye arriving in the right atrium follows a shorter pathway between the right atrium and the left atrium than the normal pathway through the lungs. The peak amplitude of the measured ICG dye concentration (relative to baseline) associated with a premature shunt curve or inflection, if present, is divided by the peak amplitude of the measured ICG dye concentration (relative to baseline) for the normal indicator-dilution curve. This ratio, expressed in percent, approximates the relative amount of ICG dye that passes through a shunt, if present, to the total amount of blood otherwise flowing through the normal pathway of the heart.

Another alternative method for the detection of the presence of a right-to-left cardiac shunt uses an injectable indicator dye in combination with a densitometer positioned at the ear of a subject. This alternative method measures the relative concentration of an injected dye as a function of time by measuring the instantaneous absorption of the dye-specific wavelength by transmitting light through the thickness of the ear. The presence of a right-to-left shunt is again determined with this method, apparatus and system by detecting the presence of a premature shunt curve or inflection that may occur in advance of the normal indicator-dilution curve associated with ICG dye following the normal pathway through the lungs. A premature shunt curve or inflection can only occur if the ICG dye arriving in the right atrium follows a shorter pathway between the right atrium and the left atrium than the normal pathway through the lungs. Regarding this shunt detection method, see:
12) Karttunen, V., et. al. “Dye Dilution and Oximetry for Detection of Patent Foramen Ovale.” Acta Neurol Scand 97:231-236 (1998).

For all four of the above shunt detection methods, it is essential that a maneuver be performed in order to increase the right-to-left pressure gradient between the right and left atria of the heart. Normally, the localized blood pressure within the left atrium is higher than the right atrium. By way of example, during normal activities that do not involve any provocations such as exertion, straining or coughing, the presence of a right-to-left shunt will result in blood flow from the left atrium of the heart to the right atrium of the heart and, accordingly, pose no risk of embolic ischemia since there is no blood flow directly from the right atrium to the left atrium across the atrial septum. However, during activities such as lifting, straining during defecation, physical sports, coughing and scuba diving, the pressure in the right atrium can briefly become larger than the pressure in the left atrium, thereby allowing a portion of the venous blood flowing through the right atrium to briefly flow directly from the right atrium to the left atrium, thereby circumventing the filtering benefit provided by the lungs.

Under the conditions of such provocations, any embolus or emboli (viz., tiny blood thrombus or thrombi) in the right atrium during the period of a positive right-to-left atrial pressure gradient can be transported directly to the left atrium. Once in the left atrium, said embolus or emboli can follow any of the normal arterial circulatory pathways which include pathways leading to the brain or the coronary arteries of the heart. Those pathways allowing any embolus or emboli to reach the brain or heart can lead to stroke or heart attack, respectively.

Several types of maneuvers have been reported that can create the required right-to-left pressure gradient to purposely induce the flow of an injected indicator or contrast agent through a right-to-left shunt, if present. Alternative maneuvers of this type include the Valsalva maneuver and coughing. The most widely used type of Valsalva maneuver is a breathing procedure involving the following three-steps: (1) inspiration (i.e., deep breath) to fill the lungs with air, (2) generation of exhalation pressure to a predetermined pressure level of about 40 mm Hg into a closed mouthpiece (usually incorporating a pressure sensing device) for a minimum period of five seconds; and (3) abrupt release of exhalation pressure followed by normal breathing.

Published clinical studies involving humans have demonstrated that a Valsalva maneuver performed according to the above three steps provides the most consistent method for inducing the right-to-left pressure gradient required to induce a temporary blood flow through any right-to-left shunt (e.g., PFO) that may be present in the heart, so as to thereby reveal the presence of said right-to-left shunt by any of the aforementioned detection methods. These published clinical studies have also confirmed that the right-to-left pressure gradient required to induce blood flow across a shunt (if present) (a) only begins upon the release or end of the Valsalva maneuver and (b) only persists for two or three heart beats or about two to three seconds following the Valsalva maneuver release. Consequently, it is critically important that the release or end of the exerted exhalation pressure occurs at the precise time period when the indicator dye or contrast agent arrives in the right atrium of the heart since the right-to-left pressure gradient persists for only two to three seconds beyond the release of the Valsalva maneuver exhalation pressure. Further background on maneuvers including Valsalva maneuvers and coughing maneuvers is found in the following articles:
13) Pfleger, S. et. al. “Haemodynamic Quantification of Different Provocation Manoeuvres by Simultaneous Measurement of Right and Left Atrial Pressure: Implications for the Echocardiographic Detection of Persistent Foramen Ovale.” Eur J Echocardiography 2: 88-93 (2001).
14) Dubourg O, Bourdarias J P, Farcot J C et al. “Contrast echocardiographic visualization of cough-induced right to left atrial shunt through a patent foramen ovale.” J Am Coll Cardiol 4: 587-594 (1984).
15) Droste D W, Kriete J U, Stypmann J et. al. “Contrast Transcranial Doppler ultrasound in the detection of right-to-left shunts: comparison of different procedures and different contrast agents.” Stroke 30: 1827-1832 (1999).

In addition to the critical timing of the release of the Valsalva maneuver exhalation pressure that is coincident with the time when an injected indicator dye agent arrives in the right atrium, it is also important to account for the differences in transit time between the site of injection (e.g., antecubital vein fossa in the arm) and the right atrium. This transit time is critical since the indicator dye or contrast agent needs to arrive at right atrium during the brief two to three second period that the right-to-left pressure gradient exists in order to cross directly into the left atrium during that brief period.

A further complication confronting methods employing indicator dye based shunt detection methods is the variability in said transit time due to differences in the venous volume in the pathway between the antecubital vein and the right atrium associated with subjects of varying size. That is, even if the indicator dye and a flushing solution is injected at a nominally constant rate, the transit time between the antecubital vein and the right atrium can vary by as much as two seconds due to vascular differences between patients. Therefore, in order to compensate for known transit time differences, it is advantageous to inject the indicator dye at two or more different time intervals (i.e., the time interval from the start of indicator injection and time of Valsalva maneuver release) in order that at least one of several selected time intervals will be appropriate to ensure that the indicator dye arrives in the right atrium during the brief period when the required right-to-left pressure gradient exists between the right and left atria.

If the indicator dye arrives too early relative to the release of the Valsalva maneuver exhalation pressure and creation of the essential right-to-left pressure gradient, then all of the dye will proceed along the normal pathway through the lungs and into the left atrium even if a right-to-left shunt is present. As a consequence, a false negative shunt test result may be returned and any existing right-to-left shunt may not be detected. Likewise, if the indicator dye arrives too late relative to the release of the Valsalva maneuver exhalation pressure, the essential right-to-left pressure gradient will have ended. Again, a false negative shunt test result may be returned.

As discussed above, the ability to detect the presence of a right-to-left shunt in the heart depends on performing a maneuver of adequate pressure intensity (viz., exhalation pressure of at least 40 mm Hg), adequate duration (viz., exhalation exertion for at least 5 seconds) and precise timing with regard to the injection of the indicator dye or contrast agent.

One known system and method for measuring exhalation pressure for the purpose of determining abdominal pressure surrounding the bladder is disclosed by de Menezes in Published U.S. Patent Application No. US2010/0234758. The system and method includes a pressure monitor with display, tubing extending from the pressure monitor to a mouthpiece and a mouthpiece. The subject exhales into the mouthpiece and the exhalation pressure level is displayed.

Another known method currently used in the conduct of Valsalva maneuvers include attaching a length of tubing to a pressure gauge or mercury manometer. The patient exhales into the tube and the exhalation pressure is dynamically displayed.

Both of the above-described methods allow the exhalation pressure to be dynamically measured. As stated above, it is essential that the pressure-producing maneuver be adequate to create a positive right-to-left atrial pressure gradient that is sufficient to induce blood flow directly from the right atrium to the left atrium (in the event a right-to-left cardiac shunt is present). In addition, it is also essential that the indicator dye or contrast agent (i.e., “indicator”) arrives in the right atrium during the brief 2 to 3 second period when the positive right-to-left pressure gradient persists so that indicator may traverse the atrial wall and reveal the presence of a right-to-left shunt.

The short time period during which the indicator dye must arrive in the right atrium is further complicated by the fact that the transit time for dye travel from the injection site (e.g., the antecubital vein at the elbow or the arm) to the right atrium depends on a number of patient specific factors. These factors include at least (a) the average lumen diameter, vein length and volume of the venous pathway between the right atrium and the injection site, and (b) the cardiac output of the patient.

To ensure that the indicator dye arrives in the right atrium in the precise window of time when the required right-to-left pressure gradient persists and to accommodate the expected, but unknown, differences in the transit time between the injection site and the right atrium, at least two tests should be performed using transit time assumptions that bracket the expected range of shortest to longest travel times from the injection site to the right atrium. By way of example, anatomical and clinical studies performed by the applicant have confirmed that two time intervals should be used between the time when the indicator dye is injected and the patient releases (i.e., ends) the Valsalva maneuver. These two time intervals have been empirically determined to be about 1.6 and 2.6 seconds.

However, clinical studies by the applicant involving over 70 patients have confirmed that the patient is not capable of consistently releasing (i.e., ending) the Valsalva maneuver precisely at a specified time interval (e.g., 1.6 and 2.6 seconds) after the time of the start of injection of the indicator dye. The inability of patients to end the Valsalva maneuver at the precise moment commanded using both visual and audible cues is due to the patient's natural response time and level of concentration during the test. This inability of the patient, for the reasons cited above, has been observed to result in variations in actual time intervals as long as 3.0 seconds beyond the intended time interval of 1.6 or 2.6 seconds. Since the response time of the patients is highly variable, no correction can be effectively applied to compensate for the natural delay associated with response to audible or visual cues.

There is, therefore, the need for a method, apparatus and system to precisely control the time interval between the detected start of indicator dye injection and the release (i.e., end) of the Valsalva maneuver when testing a patient for a right-to-left shunt as described above. There is also a need to ensure that a patient performing a Valsalva maneuver creates the required pressure using their diaphragm and not their cheek muscles, as it is known that the creation of pressure using only the cheek muscles will not create the hemodynamic conditions necessary to effect a right-to left pressure gradient between the right and left atria of the heart. There is further a need for a Valsalva mouthpiece component that can be manufactured at sufficiently low cost to enable its single and disposable use—thereby avoiding cross-contamination and pathogen transfer between patients. All of these needs are met by embodiments of the invention.

### SUMMARY

The present invention is directed, in part, to a method, apparatus and system to precisely control the time interval between the detected start of indicator dye injection and the release (i.e., end) of a Valsalva maneuver, both of which are performed during a right-to-left shunt detection test. To this end, a mouthpiece assembly is provided that comprises an ergonomic tube for insertion into the mouth, a tubular body that contains a movable shuttle that alternately isolates and exposes vent holes, an extension tube that provides hydraulic communication between the mouthpiece tubular body and a quick-disconnect fitment to enable removable attachment of the extension tubing to a mating fitment at the front panel of a controller.

The tubular body of the mouthpiece assembly may include baffle plates to direct the exhaled air away from the face of the patient when the vents are exposed at the end of the Valsalva maneuver and air is rapidly expelled from the patient's lungs. The movable shuttle component may include a pair of O-rings in combination with a biocompatible lubricant on the inner walls of the tubular body to minimize the static and dynamic friction and enable the movement of the shuttle when a negative pressure (i.e., vacuum) or positive pressure is applied by a solenoid-driven vacuum/pressurization assembly.

A solenoid-driven vacuum/pressurization assembly is provided and comprises a vacuum/pressurization body that contains a movable piston, a compression spring to return the piston to its starting position after de-energizing the solenoid, an electronically actuated solenoid, a pull rod connected between the solenoid plunger and the piston and tube support members at either end of the tubular vacuum/pressurization body to enable mounting. A pressure sensor is further provided to continuously measure the exhalation pressure exerted by a patient during performance of the Valsalva maneuver.

The shuttle within the tubular body of the mouthpiece assembly incorporates a small diameter hole that provides (a) a sufficiently large flow factor to enable pressure equalization and dynamic exhalation pressure measurement and (b) a sufficiently small flow factor to enable negative pressures (i.e., vacuum) or positive pressures (i.e., pressurization) rapidly created in the solenoid-driven vacuum/pressurization assembly to induce rapid movement of the shuttle within the mouthpiece assembly from a “vents closed” position during the period of the Valsalva maneuver to a “vents open” position at the moment of intended Valsalva maneuver pressure release.

A microprocessor of the controller receives an input via an analog/digital converter from an optical sensor that detects the start of injection of an optically opaque indicator dye, e.g., ICG dye, which is a step of the right-to-left shunt detection test. The microprocessor starts a clock and when the elapsed time is equal to a specified time interval (e.g., 1.60 or 2.60 seconds), a command is issued to a digital/analog converter to effect the actuation of a solenoid (e.g., a pull-type solenoid). The actuation of the solenoid causes the piston of the solenoid-driven vacuum/pressurization assembly to quickly retract, thereby rapidly creating a partial vacuum within the mouthpiece assembly. The partial vacuum created within the mouthpiece assembly causes the shuttle to rapidly retract from the “vents closed” proximal position to the “vents open” distal position within the tubular body of the mouthpiece assembly.

As a consequence, within a very brief period from the actuation of the solenoid valve, the opening of the vents causes a rapid release of the pressure resistance produced by the mouthpiece assembly. This forces the patient to rapidly exhale, thereby releasing (ending) the Valsalva maneuver at the desired time.

In order to accommodate the variability in the transit time between the site of indicator dye injection and the right atrium, embodiments of the present invention employs the use of two sequential tests at two different time intervals (e.g., 1.60 and 2.60 seconds). Accordingly, at the end of the first test (e.g., time interval of 1.60 seconds) and within a brief period after the mouthpiece assembly vents are opened (e.g., 5 seconds), the solenoid is de-energized and a compression spring forces the piston of the vacuum/pressurization device to rapidly return to its original position. This rapid return to the piston's original position re-pressurizes the mouthpiece assembly. As a consequence, the shuttle within the mouthpiece assembly rapidly returns to its original position, which corresponds to the vents being closed. At this stage, the mouthpiece assembly is ready for the second test procedure, viz., a test procedure at the second of the two selected time intervals (e.g., 2.6 seconds).

Embodiments of the present invention are further directed to a method of manufacture and assembly of a Valsalva maneuver mouthpiece assembly that may be cost-effectively provided in sterile condition for a single test session by a patient and then discarded. The single use of the mouthpiece assembly is preferred due to the necessary movable shuttle component within the mouthpiece assembly, the benefit to providing a lubricant on the interior of the tubular body of the mouthpiece assembly and the inaccessibility of the interior portions of the mouthpiece assembly to enable essential cleaning and sterilization of the mouthpiece assembly between uses.

Other aspects and features of the invention will become apparent to those skilled in the art upon review of the following detailed description of exemplary embodiments along with the accompanying drawing figures.

### BRIEF DESCRIPTION OF THE DRAWINGS

In addition to the features mentioned above, other aspects of the invention will be readily apparent from the following descriptions of the drawings and exemplary embodiments, wherein like reference numerals across the several views refer to identical or equivalent features, and wherein:

FIG. 1 is a partially sectioned and cut away perspective view, of an exemplary embodiment of a system showing a monitor, catheter set and mouthpiece assembly for performance of a Valsalva maneuver and controlled release of the Valsalva maneuver by a patient being tested for the presence of a cardiac shunt using an indicator dye method;

FIG. 2 is an exploded view of mouthpiece assembly of FIG. 1;

FIG. 3 is a cross-sectional view of the mouthpiece assembly showing a shuttle in initial proximal position in preparation for the start of a Valsalva maneuver;

FIG. 4 is a cross-sectional view of the mouthpiece assembly showing the shuttle in a most distal position at the release (end) of a Valsalva maneuver;

FIG. 5A is a side view of a shuttle component used in a mouthpiece assembly;

FIG. 5B is a cross-sectional view of a shuttle component used in a mouthpiece assembly;

FIG. 5C is a perspective view of a shuttle component used in a mouthpiece assembly, showing a proximal end of the shuttle;

FIG. 5D is a perspective view of a shuttle component used in a mouthpiece assembly, showing a distal end of the shuttle;

FIG. 6 is a perspective view, partly in section, of an exemplary embodiment of a vacuum/pressurization subassembly;

FIG. 7 is an exploded view of vacuum/pressurization subassembly of FIG. 6;

FIG. 8A is a cross-sectional view of an exemplary piston used in a vacuum/pressurization subassembly;

FIG. 8B is a perspective view of an exemplary piston used in a vacuum/pressurization subassembly;

FIG. 9 is a perspective view of an exemplary vacuum/pressurization tube used in a vacuum/pressurization subassembly;

FIG. 10A is a cross-sectional view of an exemplary first tube support end plate used in a vacuum/pressurization subassembly;

FIG. 10B is a perspective view of an exemplary first tube support end plate used in vacuum/pressurization subassembly;

FIG. 11A is a cross-sectional view of an exemplary second tube support end plate used in vacuum/pressurization subassembly;

FIG. 11B is a perspective view of an exemplary second tube support end plate used in vacuum/pressurization subassembly;

FIG. 12A-12E combine as labeled thereon to show a flow chart of a procedure associated with an exemplary method of the invention for performing a Valsalva maneuver as part of a indicator dye based procedure for the detection of a right-to-left shunt;

FIG. 13A-13C combine as labeled thereon to show a flow chart of a procedure associated with an exemplary embodiment of the invention for performing a Valsalva maneuver; and

FIG. 14 is a partially sectioned and cut away perspective view of an exemplary system showing a monitor and mouthpiece assembly for performance of a Valsalva maneuver and for the controlled release of a Valsalva maneuver by a patient being tested for the presence of a cardiac shunt, in general.

### DETAILED DESCRIPTION OF EXEMPLARY EMBODIMENT(S)

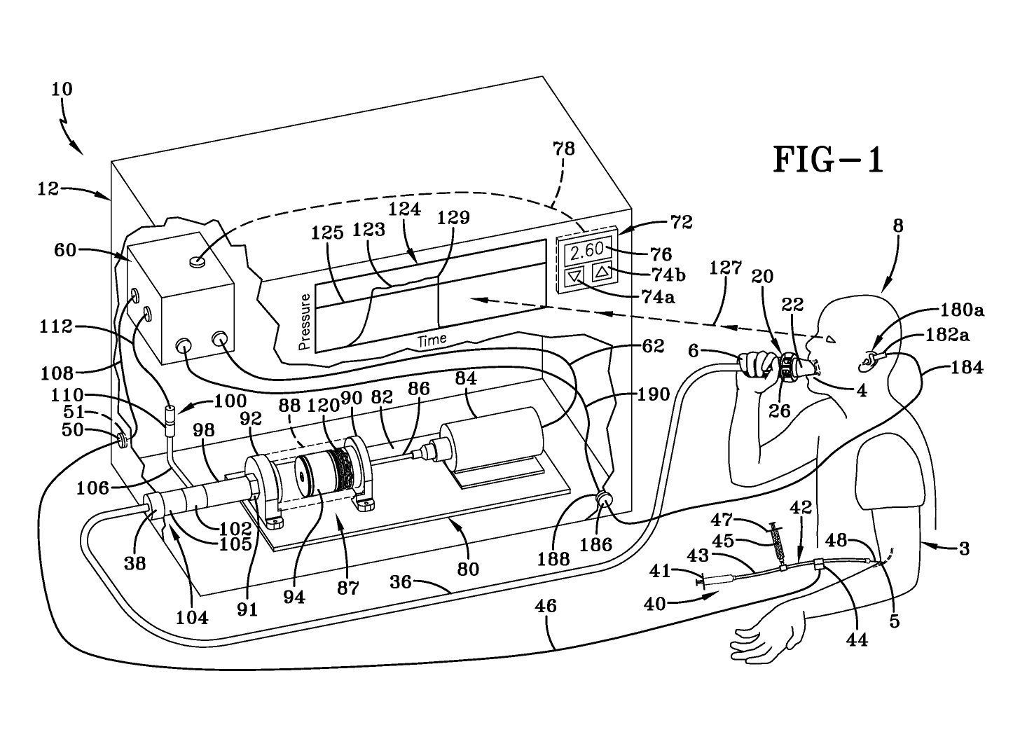

Referring to FIG. 1, an illustrative embodiment of the present invention is described showing the principal components of a system for controlling the performance of Valsalva maneuvers. As seen in FIG. 1, patient 8 grasps mouthpiece assembly 20 with hand 6 and positions ergonomic tube 22 in his or her mouth 4. By way of example as seen in FIG. 1, patient 8 is performing a Valsalva maneuver as one essential step in the detection of a right-to-left cardiac shunt based on an indicator dye method.

An exemplary embodiment of a monitor 10 for the detection of a right-to-left cardiac shunt, as seen in FIG. 1, includes a catheter set 40 for injection of an indicator dye 45 into the blood stream of patient 8 at the antecubital vein 5 of the arm 3 of patient 8. Injection of indicator dye 45 may be achieved by depressing a plunger of a dye syringe 47 containing indicator dye 45 and attached to flexible catheter 42. By depressing plunger of dye syringe 47, indicator dye 45 (e.g., liquid volume of 1 to 10 ml) is forced to flow via catheter 42 past a flow sensor 44 to a venous access needle 48 and into the antecubital vein 5 in arm 3 of patient 8.

As the indicator dye passes through flow sensor 44, it is detected using, by way of example, a measured change in the level of light transmission through the flowing liquid for the case of an indicator dye 45 that has a lower transmission of light photons than water or isotonic saline solution residing in catheter 42 prior to the start of injection of indictor dye 45. Alternatively, the indicator dye 45 that passes through flow sensor 44 may be detected by measuring a temperature decrease within a pre-heated flow sensor 44 as injected liquid induces heat removal from the heated flow sensor and an associated decrease in its measurable temperature.

Still referring to FIG. 1, immediately after indicator dye 45 has been injected, a second injection of isotonic saline may be injected via catheter 42 using a flush syringe 41 positioned at the proximal end of catheter set 40 to deliver any residual indicator dye 45 residing in catheter 42 into the blood stream of patient 8. The detection of indicator dye 45 by flow sensor 44 is communicated to a controller 60 of monitor 10 via a cable 46 that is removably connected to monitor 10 at a connector 50, which is inserted into a receptacle 51 connected to controller 60 via a cable 108.

Still referring to FIG. 1, a cut away view of an enclosure 12 of exemplary monitor 10 reveals controller 60, a solenoid-driven vacuum-pressurization assembly 80 and an internal tubing assembly 100. In one exemplary embodiment of the solenoid-driven vacuum-pressurization assembly 80, a solenoid 84 (e.g., a Pull-Type Tubular Solenoid, Ledex 150, from Johnson Controls, Vandalia, Ohio) is securely attached to a platform 82 and the plunger of solenoid 84 is mechanically coupled to a piston 94 in vacuum/pressurization subassembly 87 with a solenoid pull rod 86.

By way of example, prior to energizing solenoid 84, piston 94 is initially maintained against the inner face of a second tube support end plate 92 at the distal position within a vacuum/pressurization tube 88 due to the force applied by a compression spring 120. When solenoid 84 is energized by a power source (not shown) through controller 60 and an associated cable 62, the plunger within solenoid 84 rapidly retracts, typically within a period of less than 0.1 seconds. Upon the rapid retraction of the plunger (not shown) in solenoid 84, piston 94 rapidly moves to a fully retracted position while contracting compression spring 120 based on the allowable stroke length of the plunger in solenoid 84 and as a result of the pull force applied through solenoid pull rod 86.

The rapid retraction of piston 94 creates a negative pressure within the enclosed air space comprising vacuum/pressurization tube 88, the interior volume of an inner tubing assembly 100, the interior volume of associated extension tubing 36, and the internal volume at distal end of mouthpiece assembly 20. The negative pressure created by rapid withdrawal of piston 94 when solenoid 84 is energized causes a shuttle (not shown) within mouthpiece assembly 20 to be retracted from its starting proximal position to a distal position within a tubular body (not shown), thereby exposing a multiplicity of vent holes 26. The processes involved and the effect of the alternating negative pressure and positive pressure created by operation of the solenoid-driven vacuum/pressurization assembly 80 are described in greater detail in the discussion that follows.

Still referring to FIG. 1, the rapid retraction of the shuttle (not shown) within the mouthpiece assembly 20 results in a low flow resistance pathway between the ergonomic tube 22 in mouth 4 of patient 8 and the surrounding atmosphere external to the mouthpiece assembly 20. Said low flow resistance pathway causes patient 8 to rapidly exhaust all of the compressed air in the lungs of patient 8, thereby ending the Valsalva maneuver. Within a time period (e.g., 5 seconds) sufficient to ensure both (a) complete expiration by the patient and (b) sufficient ingress of air into the enclosed air space comprising vacuum/pressurization tube 88, interior volume of inner tubing assembly 100, interior volume of extension tubing 36 via pressure equalization conduit 18.

The latter ingress of air provides for the return of the air pressure within this air space to approximately atmospheric pressure. Following this ingress of air over a brief period (e.g., 5 seconds), solenoid 84 within solenoid-driven vacuum/pressurization assembly 80 is de-energized. Upon de-energizing solenoid 84, the magnitude of the pull force previously applied by solenoid 84 on piston 94 though solenoid pull rod 86 becomes zero. The retraction of piston 94 also induces contraction of compression spring 120.

When the pull force exerted by solenoid 84 rapidly decreases to zero as solenoid 84 is de-energized, the energy stored in compression spring 120, while in its contracted state, forces piston 94 to rapidly return to its most distal position adjacent to second tube support end plate 92. The rapid return of piston 94 to its distal position, under the force applied by compression spring 120, creates a positive pressure within the enclosed air space comprising vacuum/pressurization tube 88, interior volume of inner tubing assembly 100, interior volume of extension tubing 36 and internal volume at distal end of mouthpiece assembly 20. The positive pressure created by the rapid displacement of piston 94 to its most distal position when solenoid 84 is de-energized causes the shuttle (not shown) within mouthpiece assembly 20 to be displaced from its distal position to a proximal position within tubular body (not shown), thereby once again isolating the multiplicity of vent holes 26 in mouthpiece assembly 20 from the interior of the ergonomic tube 22 in preparation for the performance of a subsequent Valsalva maneuver.

In the above discussion of the cyclic operation of solenoid 84 in conjunction with FIG. 1, a power supply (not shown) within controller 60 applies power to solenoid 84 through power cable 62 based on a predetermined time interval, T_{In}. By way of example, said time interval, T_{In} may be electronically selected in monitor 10 at a time interval control unit 72 using manually actuatable switches 74a and 74b to effect increasing and decreasing time increments, respectively. The process of selecting the time interval, T_{In} may be accomplished with the time interval display 76 as seen in FIG. 1. The selected time interval, T_{In} is communicated to controller 60 via a cable 78. In an exemplary embodiment, the selected time interval, T_{In} is combined in controller 60 with the detected start of the injection of indicator dye 45 by flow sensor 44 to determine when solenoid 84 is to be energized, i.e., when the Valsalva maneuver is to end.

Another exemplary embodiment of the invention is the provision of a visual display of the exhalation pressure exerted by patient 8 during the Valsalva maneuver. As discussed in the Background of the Invention, prior clinical studies have confirmed that the required level of exhalation pressure exerted by a patient during a Valsalva maneuver is at least about 40 mm Hg in order to induce a right-to-left atrial pressure gradient sufficient to reveal the presence of a right-to-left shunt (e.g., a PFO). In addition, prior clinical studies have confirmed that the exertion of an exhalation pressure of at least about 40 mm Hg by the patient during a Valsalva maneuver needs to be at least 5 seconds in duration. As seen in FIG. 1, the Valsalva pressure level 123 exerted by patient 8 is visually displayed, by way of example, in the form of a real-time graph as seen by patient 8 at Valsalva screen display 124 of monitor 10. In the example Valsalva pressure screen display 124 seen in FIG. 1, a horizontal line representing the minimum required Valsalva pressure level 125 provides visual feedback to patient 8 to guide their exertion level during the Valsalva maneuver.

In actual practice, monitor 10 is preferably positioned such that the Valsalva pressure screen display 124 is in the direct line-of-sight of patient 8. However, to facilitate the illustration of all of the components of monitor 10, mouthpiece assembly 20 and catheter set 40 in FIG. 1, the Valsalva pressure screen display 124 is not in the line-of-sight of patient 8 but said line-of-site is represented by sighting path 127.

In the example graph of exhalation pressure as a function of time seen in Valsalva pressure screen display 124 of FIG. 1, the Valsalva maneuver has been completed and the Valsalva release 129 is seen at the end of the period of exhalation exertion corresponding to the moment when the shuttle (not shown) in mouthpiece assembly 20 is rapidly translated to said distal position by the negative pressure rapidly induced by solenoid-driven vacuum/pressurization unit 80, thereby exposing one or a multiplicity of vents 26 and, thereby, forcing complete expiration of the air within the lungs of patient 8. This complete expiration of the air within the lungs of patient 8 represents the release or end of the Valsalva maneuver.

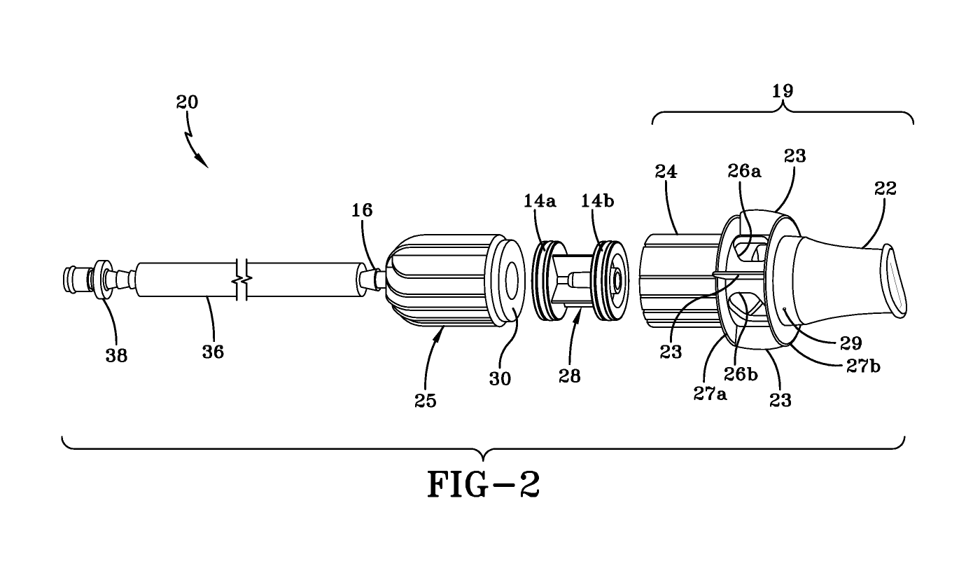

Referring now to FIG. 2, an exploded view of one exemplary embodiment of mouthpiece assembly 20 is shown in greater detail, and can be seen to comprise an ergonomic tube 22, tubular body 24, shuttle 28 and end cap 25. A compliant foam rubber sleeve (not shown) may optionally be positioned over tubular body 24 and end cap 25 to facilitate grasping of mouthpiece assembly 20 in either the left or right hand 6 of a patient as illustrated by patient 8 in FIG. 1. Still referring to FIG. 2, an exemplary embodiment of a subassembly 19 may be injection or otherwise molded using a suitable biocompatible plastic offering a relatively low coefficient of friction relative to shuttle O-rings 14a and 14b, and also offering good dimensional control through the injection molding process. By way of example, one usable injection moldable plastic for subassembly 19 is acrylonitrile butadiene styrene (ABS) or blends containing ABS, such as those manufactured by Bayer AG (distributed through Bayer USA, Pittsburgh, Pa.).

The subassembly 19 of this embodiment comprises ergonomic tube 22, tubular body 24, one or more vent holes 26, first and second baffle plates 27a and 27b, radial ribs 23a-23d and a leak hole 29. The circular bore of tubular body 24 is accurately dimensioned to receive shuttle 28, including first and second shuttle O-rings 14a and 14b. Embodiments of shuttle 28 may be injection molded using a suitable plastic offering good dimensional control through the injection molding process.

Radial ribs 23 in combination with first and second baffle plates 27a and 27b prevent the hand 6 of a patient from grasping and covering over one or more vent holes 26 and, thereby, causing interference with the air flow exiting the vents when shuttle 28 is translated to its distal position (i.e., the “vents open” position). As seen in FIG. 2, first and second baffle plates 27a and 27b are positioned on subassembly 19 just distal to ergonomic tube 22. During use, as illustrated in FIG. 1, the ergonomic tube 22 is placed in the mouth 4 of patient 8. Upon the forward translation of the shuttle 28 to its most distal position and exposure of one or more vents 26, said baffle plates 27a and 27b serve to direct the rapidly expelled air (issuing from the lungs of patient 8) away from the face and eyes of patient 8.

Still referring to FIG. 2, the interior circular bore of tubular body 24 is dimensioned so that the static friction between shuttle O-rings 14a and 14b and interior wall of tubular body 24 is (a) sufficiently large such that exhalation pressure exerted by patient 8 does not prematurely translate shuttle 28 to a distal position that exposes a portion or all of the one or more vents 26 yet (b) sufficiently small that the negative pressure induced by displacement of the piston in the solenoid-driven vacuum/pressurization assembly creates the force necessary to rapidly translate the shuttle from its starting (proximal) position in which it covers one or more vents 26 to its distal position exposing one or more vents 26.

In one exemplary embodiment and still referring to FIG. 2, and end cap 25 is inserted into and adhesively bonded to tubular body 24 to provide a seal to prevent the egress of air during the Valsalva maneuver. This particular end cap 25 also includes a noise-dampening elastomeric washer 30 to absorb and dissipate the impact energy associated with the rapid translation of shuttle 28 to its most distal position, thereby reducing the noise associated with the translation of shuttle 28 to the distal end of tubular body 24. Also, as seen in the exemplary embodiment shown in FIG. 2, end cap 25 includes a barbed fitment 16 for airtight attachment of the proximal end of flexible extension tube 36 to end cap 25. The distal end of extension tube 36 is secured to a similar barbed fitment at the proximal end of quick disconnect fitment 38. Such a quick disconnect fitment is available from Colder Products Company, Minneapolis, Minn.

By way of example with respect to the embodiments shown in FIGS. 1 and 2, said extension tubing 36 is a biocompatible flexible vinyl tubing having an inside diameter of 0.187 inch and length of 48 inches (Cole-Parmer, Vernon Hills, Ill.). The inside diameter of extension tubing is selected to be (a) large enough to enable sufficient air flow and associated rapid evacuation of air from the distal end of tubular body 24 when piston 94 of solenoid-driven vacuum/pressurization assembly 80 is rapidly withdrawn by energized solenoid 84 and (b) small enough that the interior volume of extension tubing 36, in combination with the interior volume of interior tubing set 100 and the end of tubular body 24 distal to shuttle 28, are sufficiently small to enable an adequate negative pressure within combined total interior volume to force translation of shuttle 28 when said solenoid 84 in solenoid-driven vacuum/pressurization assembly 80 is energized.

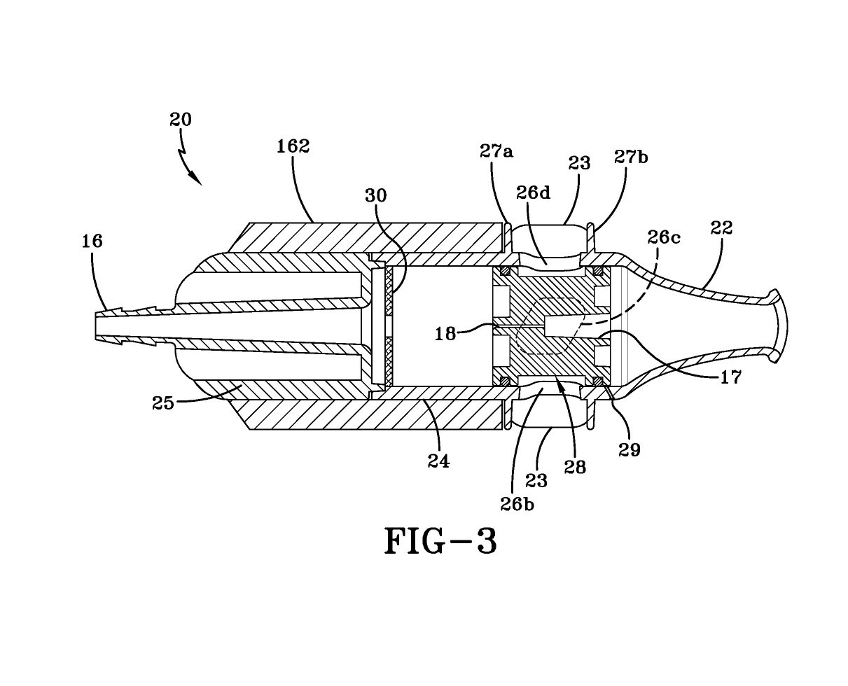

An assembly view of an exemplary embodiment of the mouthpiece assembly 20 is depicted in FIG. 3 and FIG. 4 following the placement of shuttle 28 within tubular body 24 and attachment of end cap 25. A compliant sleeve 162 (e.g., biocompatible foam rubber) surrounds and is secured to tubular body 24 and end cap 25 to facilitate grasping of mouthpiece assembly by hand 6 of patient 8 as seen in FIGS. 1, 3 and 4. Also revealed in FIGS. 3 and 4 is noise dampening elastomeric washer 30 mounted on proximal interior surface of end cap 25. An exemplary embodiment of end cap 25 may be injection molded using a suitable biocompatible plastic. By way of example, a suitable injection moldable plastic for end cap 25 is acrylonitrile butadiene styrene (ABS) or blends containing ABS, such as those manufactured by Bayer AG (distributed through Bayer USA, Pittsburgh, Pa.).

Referring now to FIG. 3, shuttle 28 is seen in its initial proximal position in preparation for the start of the Valsalva maneuver. In this proximal position, the shuttle 28 remains stationary during the pressure exertion period of the Valsalva maneuver and blocks air flow access to one or more vents 26 so that patient 8 is able to perform Valsalva maneuver by exerting exhalation pressure of about 40 mm Hg from his or her lungs into ergonomic tube 22 and into the essentially closed volume at the end of the tubular body 24, extension tubing 36 and internal tubing assembly 100. A small leak hole 29 is located proximal to first shuttle O-ring 14a to allow a small flow rate of air to escape from mouthpiece assembly 20 during the Valsalva maneuver.

By way of example and through clinical testing with human subjects, it has been determined that a leak rate of about 20 to 25 cubic centimeters per second under an applied exhalation gauge pressure of 40 mm Hg is (a) large enough to ensure that the exhalation pressure must be exerted by the lungs of patient 8 and not through the use of contraction of distended cheek muscles and (b) small enough to enable an adult to maintain an exhalation pressure of about 40 mm Hg for a period of at least 5 seconds without depleting their natural lung volume capacity. Also, FIG. 3 reveals enlarged entrance hole 17 and pressure equalization channel 18 within shuttle 28, which enables exhalation pressure exerted during the Valsalva maneuver by patient 8 to be dynamically measurable by pressure sensor 110 (see FIG. 1) by virtue of the air column between the mouthpiece assembly 20 and pressure transducer 110.

Referring now to FIG. 4, the shuttle is seen it its most distal position corresponding to the period immediately following the negative pressure induced by the retraction of piston 94 in the solenoid-driven vacuum/pressurization assembly 80 (see also FIG. 1). As seen in FIG. 4, the translation of shuttle 28 to its most distal position exposes one or more vents 26 to the surrounding atmospheric pressure conditions. Immediately following the exposure of the vents and the associated low air flow resistance pathway between ergonomic tube 22 and one or more vents 26, the exhalation exertion by patient 8 ends with the rapid expiration of all pressurized air within the lungs. The rapid expiration of all pressurized air within the lungs thereby ensures the end of the Valsalva maneuver at the precise time interval, T_{In selected by the operator at time interval control unit 72.}

Referring to FIGS. 2, 3 and 4, the shape of the opening of said one or more vents 26 may be of various shape, such as circular, trapezoidal or square. In an exemplary embodiment, the shape of the opening of six vents 26a-26f may be circular or trapezoidal to minimize the friction between the proximal shuttle O-ring 14a as it traverses the perimeter edges of vents 26a-26f. Also, to ensure acceptably low static and dynamic friction to enable translation of the shuttle from its proximal position as seen in FIG. 3 to its distal position as seen in FIG. 4, a biocompatible lubricant (Dow Corning Silicone 360 Lubricant, Midland, Mich.) is preferably applied (not shown) to the inner smooth walls of tubular body 24 and shuttle O-rings 14a and 14b.

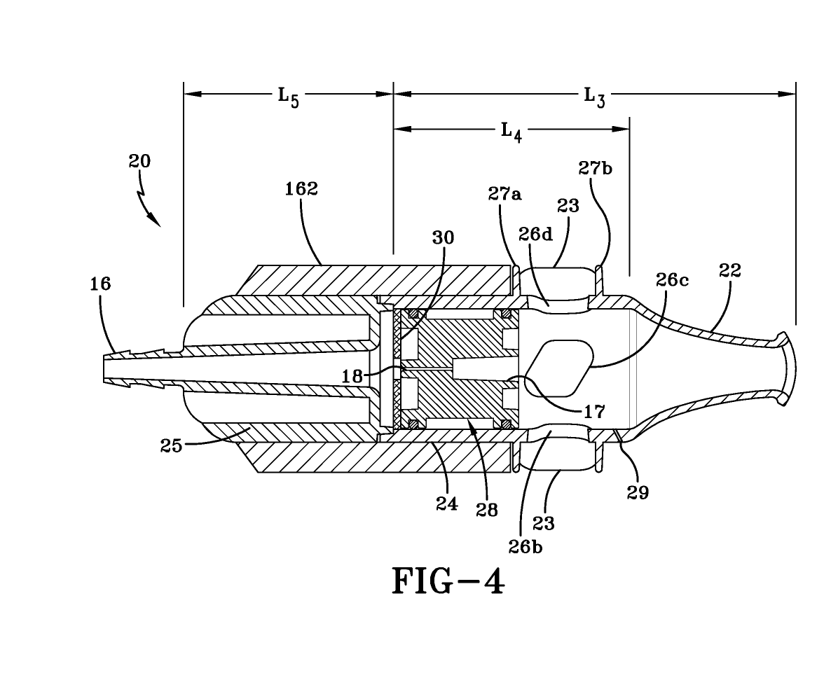

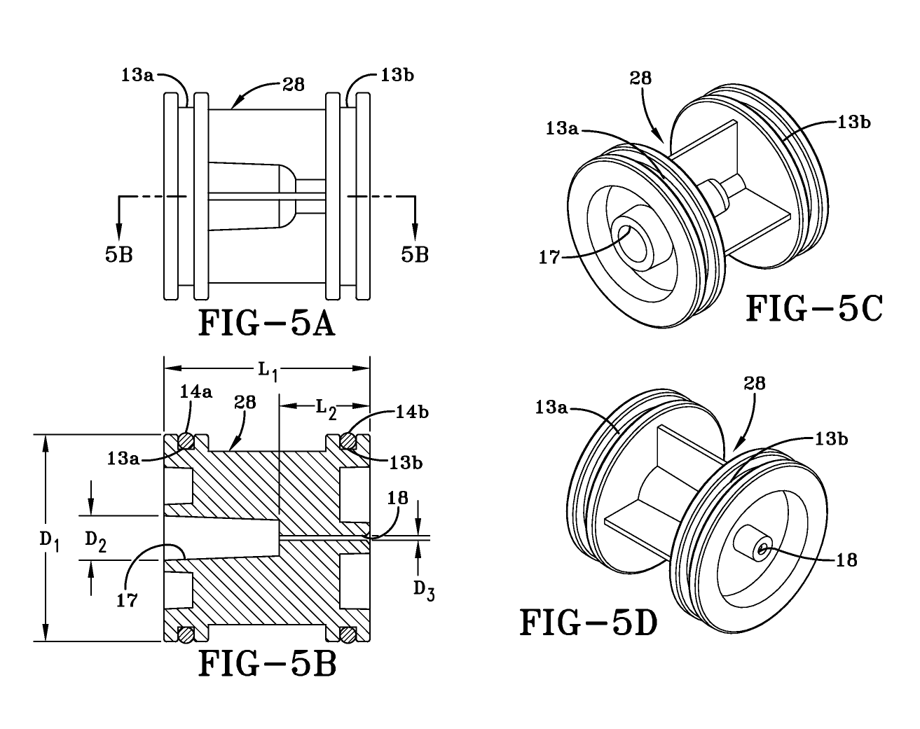

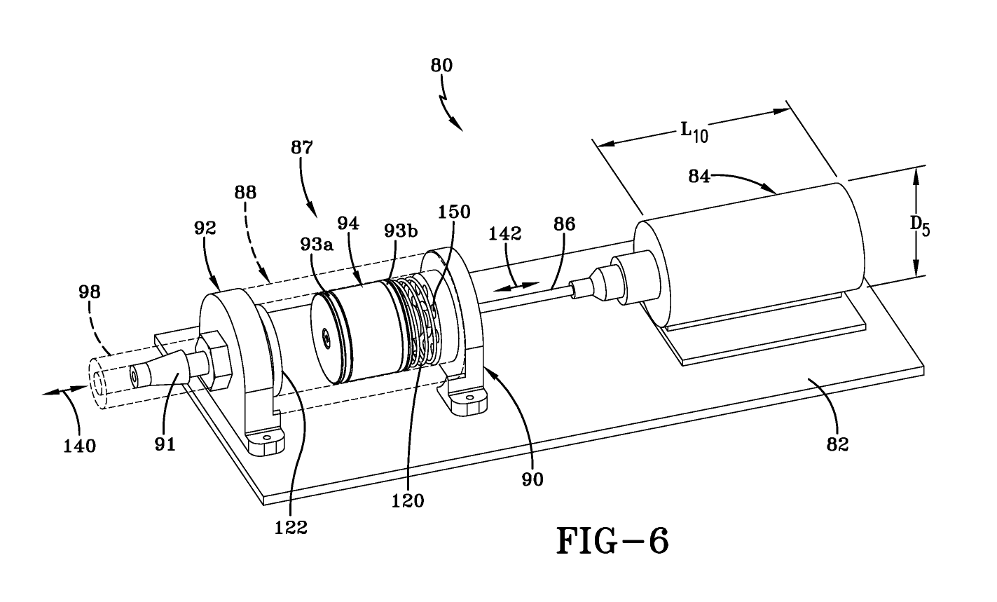

Referring now to FIGS. 5A through 5D, shuttle 28 is seen in a side view, cross-sectional view and perspective views. As can be seen in the side view of FIG. 5A, shuttle 28 includes shuttle O-ring grooves 13a and 13b. The cross-sectional view seen in FIG. 5B reveals elastomeric shuttle O-rings 14a and 14b positioned in shuttle O-ring grooves 13a and 13b. By way of example, shuttle O-rings 14a and 14b may be Size No. 20, Buna-N material, 0.864 inch ID×0.070 inch wide (available Parker Hannifin Corporation, Lexington, Ky.). As also seen in FIG. 5B, both a larger and smaller hole extend across the full length of shuttle 28. The larger entrance hole 17 (e.g., 0.18 inch diameter by 0.56″ long) provides a low resistance to air flow between the proximal surface of shuttle 28 and the start of the smaller diameter pressure equalization hole 18 as seen in FIG. 5B. The larger entrance hole 17 also minimizes the possibility that any fluid that might be ejected from the mouth 4 of patient 8 results in the occlusion of the pressure equalization channel 18. An exemplary embodiment of shuttle 28 of the invention includes a pressure equalization channel having a diameter of 0.026 inches and a length of 0.44 inches. Perspective views of shuttle 28 are seen in FIGS. 5C and 5D, revealing an opening of larger entrance hole 17 and pressure equalization channel 18, respectively.

An assembly view of the exemplary solenoid-driven vacuum/pressurization assembly 80 is illustrated in FIG. 6, which comprises vacuum/pressurization subassembly 87, solenoid 84, solenoid drive rod 86 and platform 82. Said vacuum/pressurization subassembly 87 seen in FIG. 6 comprises vacuum/pressurization tube 88, piston 94, compression spring 120 and first and second tube support endplates 90 and 92.

By way of example, an exemplary embodiment of the solenoid-driven vacuum/pressurization assembly 80 employs a Ledex 150 pull-type tubular solenoid (Johnson Controls, Vandalia, Ohio) for solenoid 84, providing a maximum stroke length of 0.7 inches and a pull-force of about 5 to 7 pounds. Still referring to FIG. 6, compression spring 120 of the exemplary vacuum/pressurization subassembly 87 may be, by way example, a stainless steel spring, having a 1.218 inch OD, a 0.063-inch wire diameter, and an overall free length of 1.75 inches (available from, e.g., Lee Spring, Bristol, Conn.). Also by way of example, vacuum/pressurization tube 88 may be machined from a plastic having a low coefficient of friction, such as acetal resin (e.g., Delrin, DuPont, Parkersburg, W. Va.), to enable reliable translation of the piston 94 within vacuum/pressurization tube 88 during alternating evacuation and pressurization cycles.

The inner circular walls of vacuum/pressurization tube 88 are preferably machined and polished to a smooth finish in order to minimize static and dynamic friction between first and second piston O-rings 93a and 93b and the inner wall of vacuum/pressurization tube 88 during the cyclic translation of piston 94. In addition, a lubricant is preferably applied to the inner walls of the vacuum/pressurization tube 88 in order to further minimize static and dynamic friction during the cyclic translation of the piston. By way of example, said lubricant (not shown) may be Super-O-Lube (Parker Hannifin Corporation, Lexington, Ky.).

As seen in FIG. 6, first and second tube support end plates 90 and 92 are attached at either end of vacuum/pressurization tube 88 with an air-tight sealing adhesive used at the interface between the vacuum/pressurization tube 88 and second tube support end plate 92. A barbed fitment 91 is attached to the exterior side of second tube support end plate 92 to provide for secure and airtight connection to first tubing member 98. Tubing member 98 extends to and is secured with an airtight seal to a “T” shaped barbed fitment (not shown) with (a) first remaining branch of the “T” extending to pressure sensor 110 via second tubing member 106 with airtight seals at both ends of tubing member 106 and (b) second remaining branch of the “T” extending to a quick-disconnect front panel receptacle 104 via third tubing member 105 with airtight seals at both ends of tubing member 105 (also refer to FIG. 1). Also seen in FIG. 6 is solenoid pull rod 86 with a movement vector 142 illustrating translation of solenoid pull rod 86 during alternating evacuation and pressurization cycles. The vacuum/pressurization subassembly 87 and solenoid 84 are mounted (e.g., mechanically attached using machine screws and nuts) on platform 82 to maintain and stabilize their relative positions during alternating evacuation and pressurization cycles. Still referring to FIG. 6, a noise dampening elastomeric disk 122 is positioned at the distal end of vacuum/pressurization tube 88 to dissipate the kinetic energy and force associated with the translation of piston 94 by compression spring 120 immediately following de-energizing of solenoid 84, thereby reducing the noise associated with the return of piston 94 to the distal end of vacuum/pressurization tube 88.

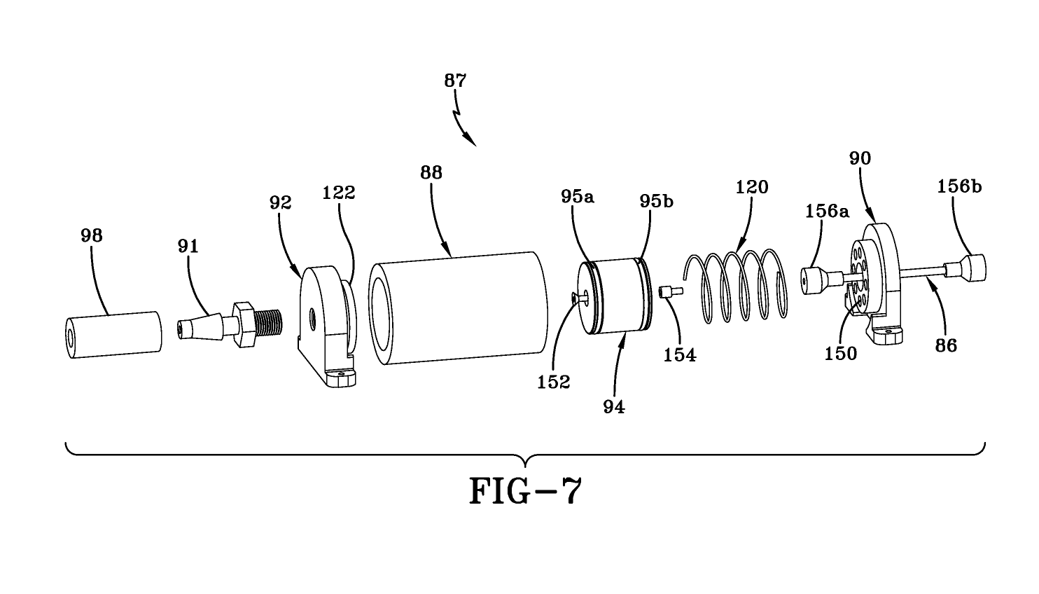

An exploded view of vacuum/pressurization subassembly 87 is seen in FIG. 7 providing addition details of an exemplary embodiment of its construction. Flat-head machine screw 152 extends through piston 94 and is threaded into piston attachment cap 154. Piston attachment cap 154 is mechanically secured to solenoid pull rod 86 at first drive rod coupling 156a. Second drive rod coupling 156b is mechanically secured to plunger (not shown) of solenoid 84.

By way example, solenoid pull rod 86 comprises a flexible cable with drive rod couplings 156a and 156b secured at either end through mechanical swaging of couplings onto flexible cable. The use of a flexible cable in solenoid pull rod 86 compensates for any misalignment that may exist between the central axis of translation of piston 94 and the central axis of translation of the plunger in solenoid 84. See for example commercially available Flexible Drive Shaft (Stock Drive Components/Sterling Instrument, New Hyde Park, N.Y.).

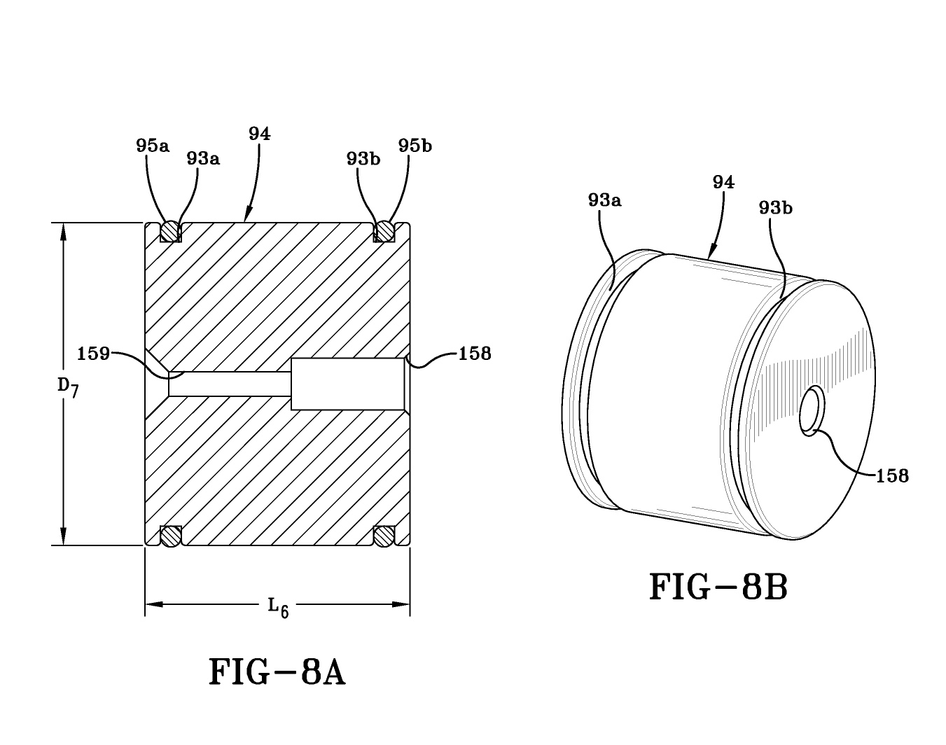

Referring now to FIGS. 8A and 8B, piston 94 is shown in a cross-sectional view and perspective view, respectively. As seen in the cross-sectional view of piston 94 in FIG. 8A, piston O-rings 95a and 95b (e.g., Buna N, Parker Hannifin Corporation, Lexington, Ky.) are positioned in piston O-ring grooves 93a, 93b. Counter bore 158 in piston 94 receives piston attachment cap 154 at the proximal end of piston 94. Drilled and counter bored hole 159 receives piston attachment flat head machine screw 152.

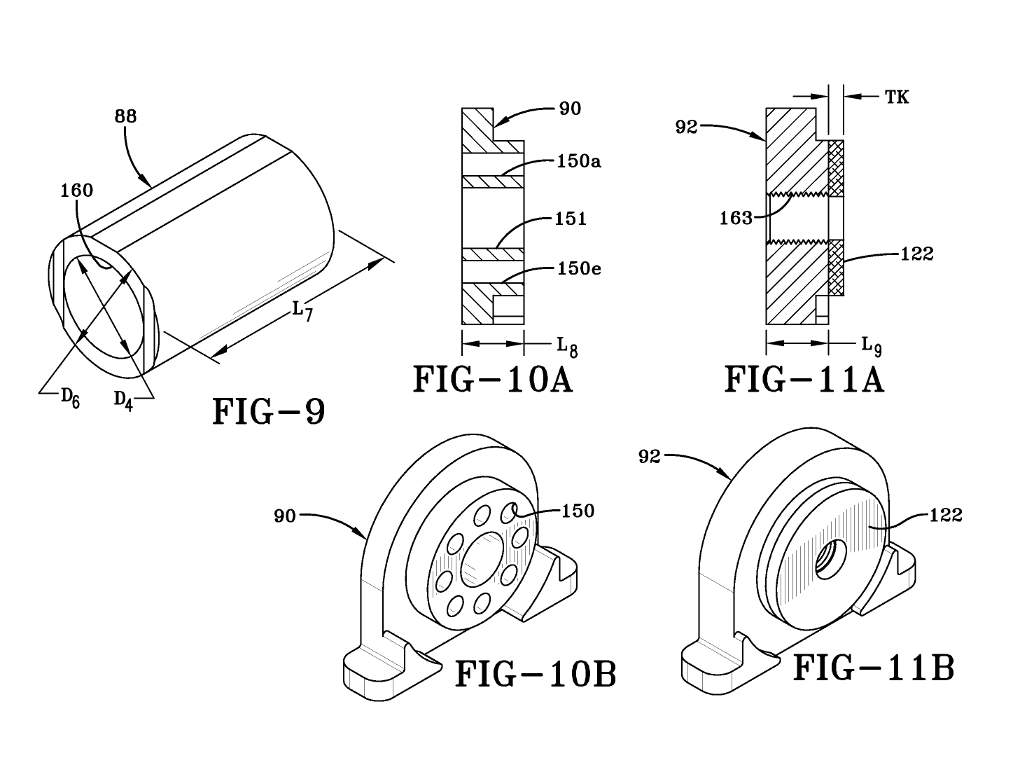

Three of the components of the exemplary embodiment of the vacuum/pressurization subassembly 87 seen in FIG. 6 are shown in greater detail in FIGS. 9, 10A, 10B, 11A and 11B. By way of example and referring first to FIG. 9, vacuum/pressurization tube 88 is shown along with defining dimensional parameters of a circular cross-section tube machined from a low coefficient of friction material (e.g., Delrin). The inner bore 160 is preferably polished to reduce the static and dynamic friction relative to piston O-rings 95a and 95b during the translation of piston 94 during the evacuation and pressurization cycles (see also FIG. 8A).

First tube support end plate 90 is seen in FIG. 10A showing circular channel 151 sufficiently large to accommodate passage of solenoid drive rod 86 and its end fitments 156a and 156b to effect a linkage between a plunger (not shown) in solenoid 84 and piston 94 (see also FIG. 7). By way of example and referring next to cross-sectional and perspective views seen in FIGS. 10A and 10B, additional vent holes 150 are machined through first tube support end plate 90 to provide a low resistance pathway for air flow into or out of vacuum/pressurization tube 88 during the translation of piston 94 associated with the evacuation and pressurization cycles (see also FIGS. 6 and 7).

By way of example and referring next to the cross-sectional and perspective views seen in FIGS. 11A and 11B, second tube end plate support 92 is covered with noise dampening elastomeric disk 122 to dissipate the kinetic energy of and force applied to piston 94 as it is translated to its most distal position by compression spring 120 following the de-energizing of solenoid 84. Threaded hole 163 is machined through the full thickness of second tube end plate support 92 to receive the threaded end of vacuum/pressurization barbed fitment 91 (see also FIGS. 6 and 7).

By way of example, the dimensions of the components of one exemplary embodiment of mouthpiece assembly 20 and solenoid-driven vacuum/pressurization assembly 80 are summarized below, in units of inches, with the identification of these dimensions seen in FIGS. 4, 5B, 6, 8A, 9, 10A and 11A. The dimensions listed below are provided merely for illustration and not limitation, as a wide range of possible dimensions would enable a functioning device as long as the vacuum and pressurization parameters as well as pressure equalization flow parameter required for reliable translation of shuttle 28 are achieved.

### TK=0.05 to 0.20

### D1=0.98

### D2=0.18

### D3=0.026

### D4=1.235

### D5=1.25

### D6=1.71

### D7=1.229

### L1=1.02

### L2=0.44

### L3=3.00

### L4=1.65

### L5=1.53

### L6=1.00

### L7=2.75

### L8=0.50

### L9=0.50

### L10=2.50

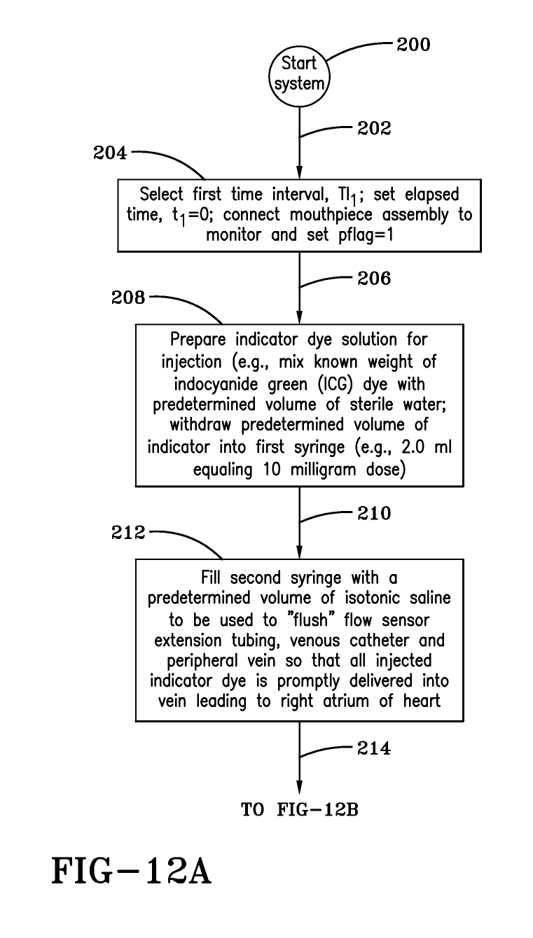

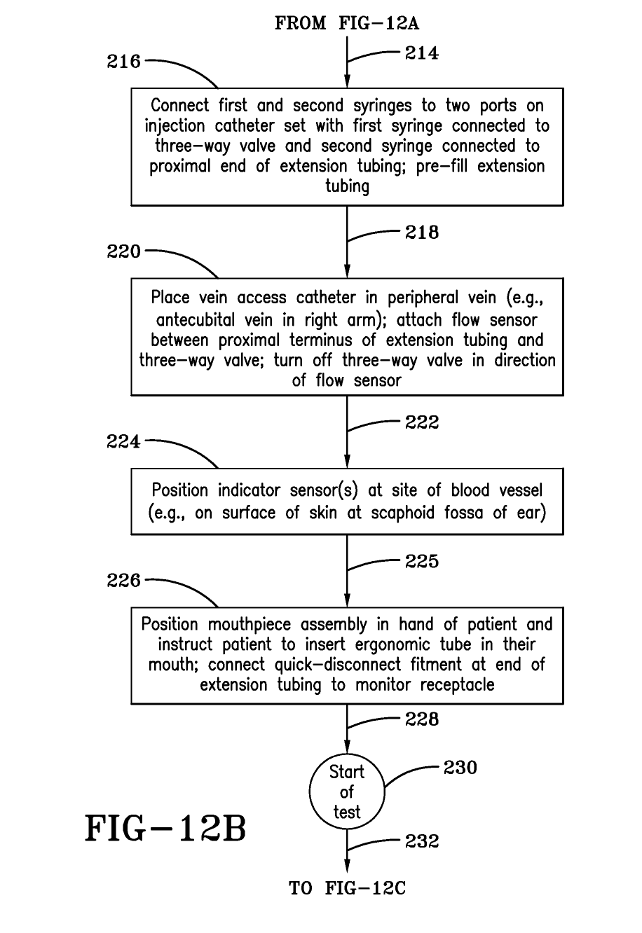

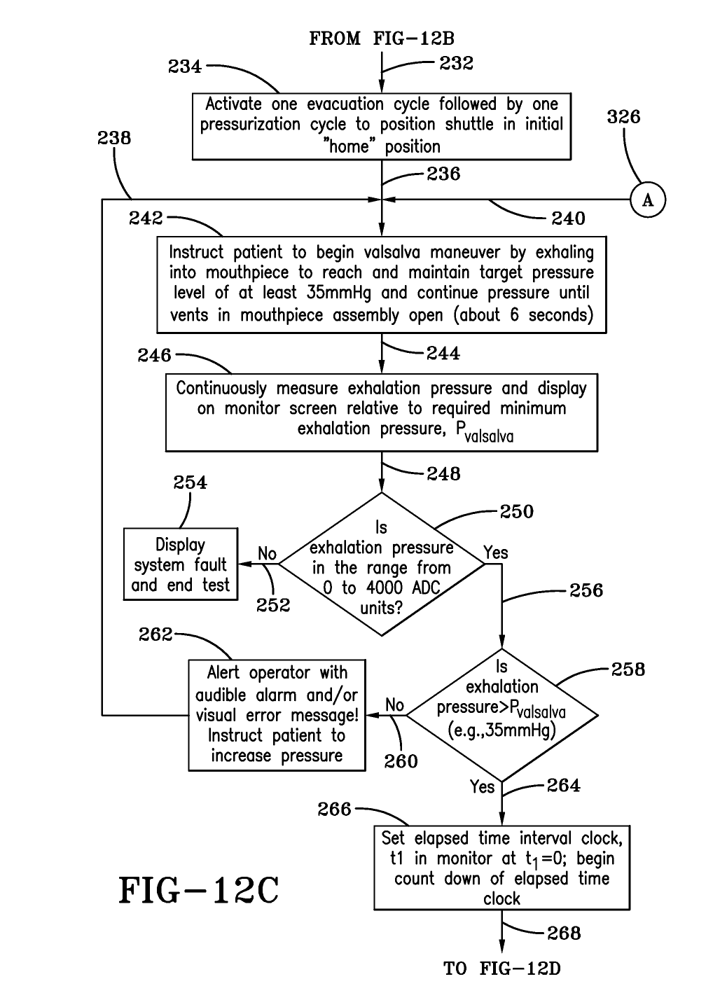

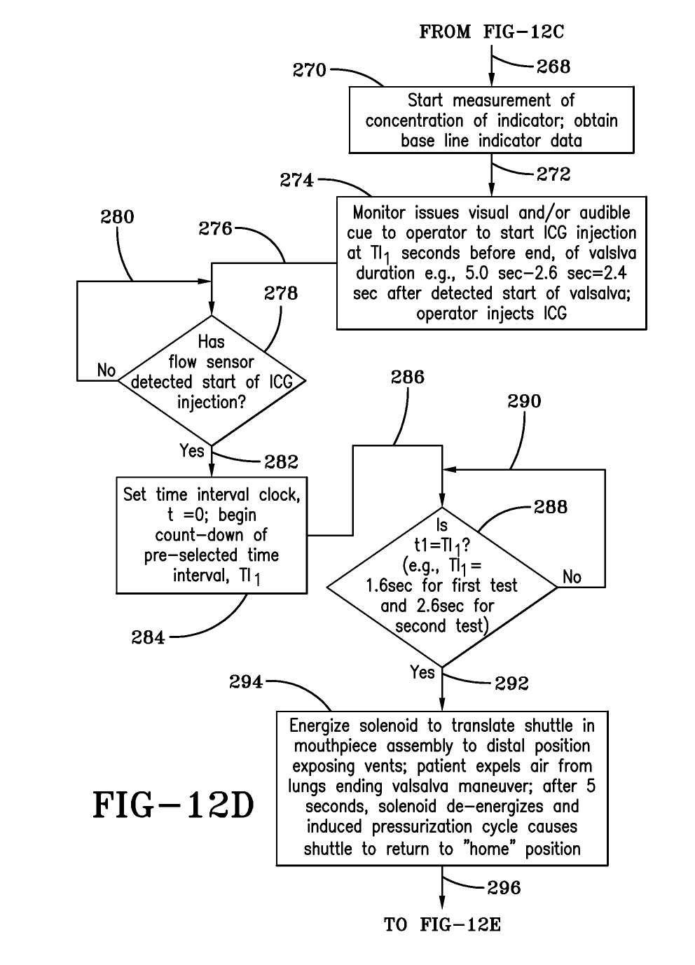

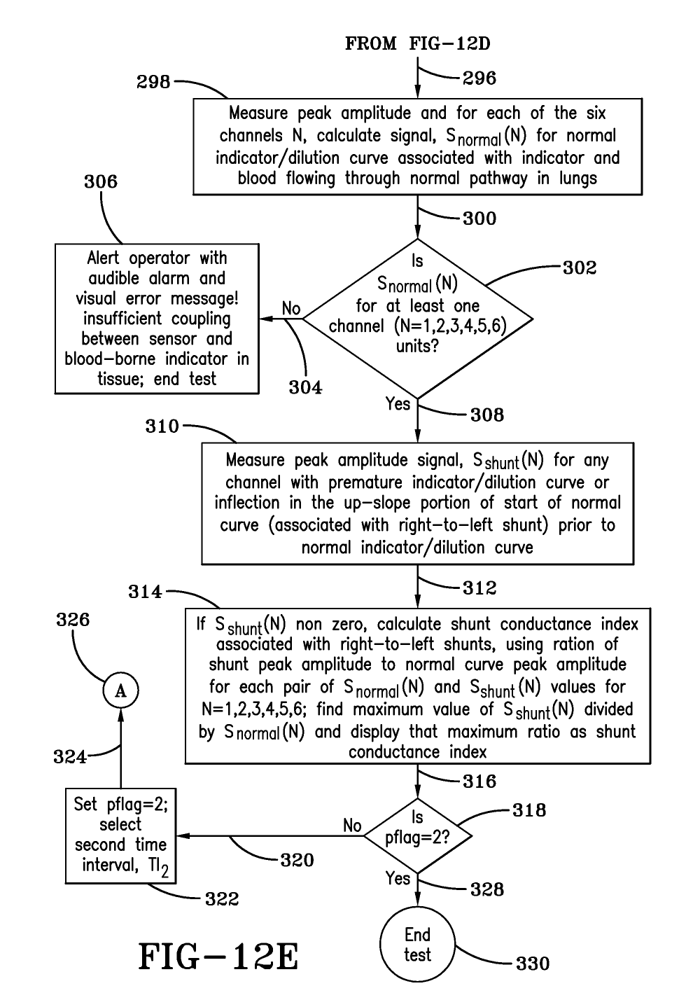

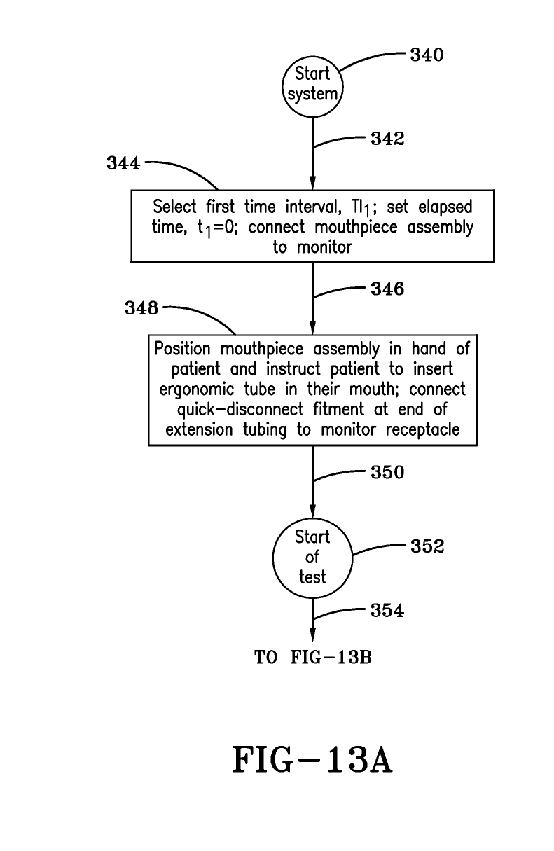

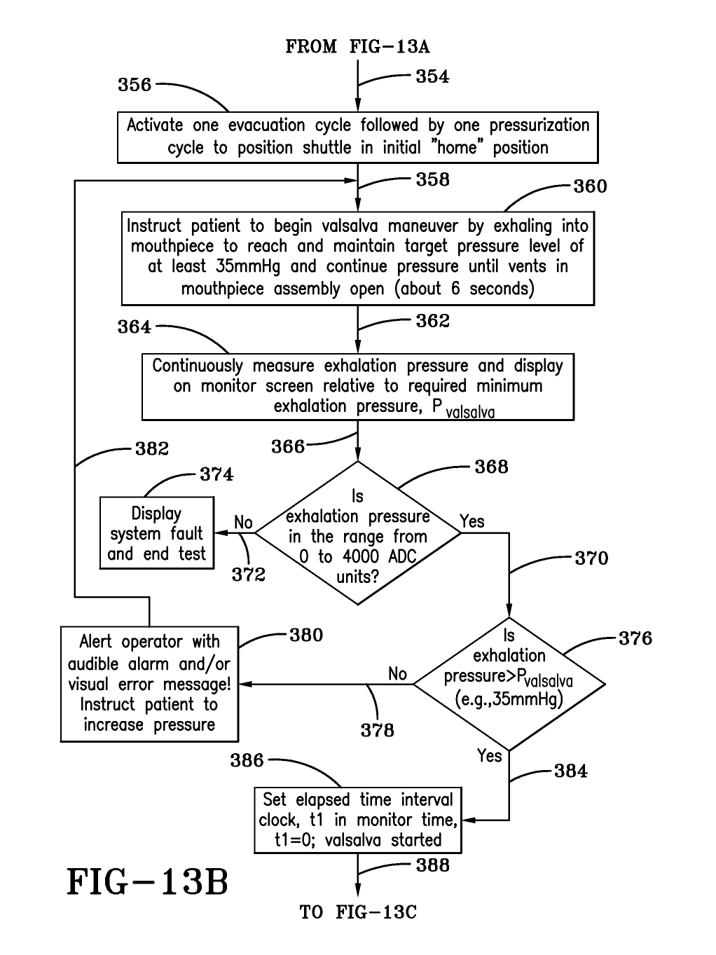

A general flow chart of the operation of an exemplary embodiment of the system is collectively represented by FIGS. 12A-12E. These figures are combined as labeled thereon to provide a single flow chart describing the exemplary system and method for the performance of a Valsalva maneuver as one of the steps of a procedure for the detection of a right-to-left cardiac shunt. The specified system for performing a Valsalva maneuver and detection of a cardiac right-to-left shunt can utilize the following protocol without extensive experimentation. The system, apparatus and method for detection of a cardiac right-to-left shunt are also described in PCT application No. PCT/US11/31433 and U.S. patent application Ser. No. 12/754,888, as mentioned previously.

Beginning as represented by symbol 200 and continuing as represented by arrow 202 to block 204, the controller carries out system initialization with the establishment of default parameters. First Time Interval, TI₁ is selected, procedure count parameter, PFLAG is set to a value of 1 and the elapsed time, t₁ is set to zero. Next, as represented at arrow 206 and block 208, the program continues where the indicator solution for injection is prepared, for example by mixing a known weight of indicator, e.g., ICG dye, with a predetermined volume of sterile water. A predetermined volume of that mixed indicator is withdrawn into a first syringe. Such a syringe is shown as 45 in FIG. 1.

The program continues as represented at arrow 210 to block 212. Block 212 provides for filling a second syringe with a predetermined volume of isotonic saline. That isotonic saline is used to “flush” the flow sensor, extension tubing, catheter, peripheral vein, and the like, so that all of the injected indicator is promptly delivered into the vein leading to the right atrium of the patient. As represented at arrow 214 to block 216 of FIG. 12B, the first syringe is connected to a three-way valve and the second syringe is connected to the proximal end of the extension tubing, which is in turn connected to a second port on the three-way valve. The three-way valve setup has been described in more detail in connection with FIG. 1. As represented at block 216, the indicator solution from the first syringe is injected into the extension tubing that is in turn connected to the three-way valve, in order to pre-fill the extension tubing with indicator solution.

Still referring to FIG. 12B, the program continues as represented at arrow 218 to block 220 to provide for placing the vein access catheter in a peripheral vein, preferably in the antecubital vein 5 of one of the arms 3 of the patient 8 as seen in FIG. 1. The flow sensor is also attached at block 220 between the proximal terminus of the extension tubing and the three-way valve as seen at 44 in FIG. 1. The three-way valve is turned off in the direction of the flow sensor.

One or more indicator sensors 182 are then positioned at a blood vessel site at arrow 222 to block 224. For example, as seen in FIG. 1, a first indicator sensor 182a may be positioned at the surface of the scaphoid fossa of the left ear 180a of patient 8 and a second indicator sensor 182b (not shown) may be positioned at the surface of the scaphoid fossa of the right ear 180b (not shown) of patient 8.

The program continues as represented at arrow 225 to block 226 to provide for placement of mouthpiece assembly in the hand of the patient and instructs the patient to place the ergonomic tube 22 in his or her mouth 4 as seen at 20 in FIG. 1. Still referring to FIG. 12B, from block 226, arrow 228 leads to the “Start of Test” indication at block 230.

Arrow 232 reappears in FIG. 12C extending to block 234, to provide for initialization of the shuttle location in the mouthpiece assembly by activating one evacuation cycle followed by one pressurization cycle to position shuttle in the initial “home” position within the tubular body of the mouthpiece assembly. Next, the program starts measurement as represented at arrow 236 extending to block 242, wherein instructions are provided to the patient to begin the Valsalva maneuver by exhaling into the ergonomic tube of the mouthpiece assembly, as seen in FIG. 1, to reach and maintain the target pressure level until the monitor terminates the Valsalva maneuver automatically after the specified Time Interval, TI₁ has elapsed.

Generally, the Valsalva maneuver procedure is accompanied by some form of display on monitor 10. By way of example, turning momentarily back to FIG. 1, a line graph 123 is provided along with a minimum exhalation pressure level 125, represented as a solid line, giving the patient the actual real-time measurement of the pressure being exerted by the patient during the Valsalva maneuver. The graph display 124 shows exhalation pressure versus elapsed time. In FIG. 1, the patient's Valsalva exhalation pressure has just been released automatically by the combined operation of the solenoid-driven vacuum/pressurization assembly and mouthpiece assembly seen at release time point 129 in FIG. 1. As seen at display screen 124 of FIG. 1, the Valsalva maneuver was properly ended with the graph 123 displaying that the patient held the proper pressure (with some acceptable variation) during the duration of the Valsalva maneuver.

Returning to FIG. 12C, as represented at arrow 244 to block 246, the exhalation pressure created by the patient during the Valsalva maneuver is continuously measured and displayed on the monitor, as explained in connection with FIG. 1, and is compared to the ideal Valsalva curve or required minimum exhalation pressure level. As represented by arrow 248 to block 250, the exhalation pressure is queried and it is determined whether it falls within a measurable range, for example from 0-4000 analog-to-digital converted (ADC) units. If not, arrow 252 is followed to block 254, wherein a system fault is displayed and the test is ended. If the measured exhalation pressure is within an expected range, arrow 256 is followed to block 258.

Still referring to FIG. 12C, block 258 poses the query as to whether the exhalation pressure is above or equal to the targeted pressure, for example 35 mm Hg. In the event that it is not, as represented at arrow 260 and block 262, the operator is alerted with an audible alarm or visual error message to instruct the patient to increase pressure to meet or exceed the target exhalation pressure level. Where the exhalation pressure is appropriate, the program continues as represented at arrow 264.

As represented at arrow 264, extending from the query at block 258 and leading to block 266, the Time Interval elapsed time clock, t₁ is set to t₁=0 and begins the countdown (i.e., count up) to the specified Time Interval value, TI₁.

Arrow 268 reappears in FIG. 12D extending to block 270, which looks to initiating the start of the measurement of the fluorescence signal level associated with the relative concentration of the injected indicator dye including obtaining the baseline signal level data (i.e., measured background signal level prior to the presence of injected indicator dye in the bloodstream as measured at the indicator sensor location) using one or more indicator sensors as seen at 182a placed on ear 180a of patient 8, as seen in FIG. 1.

As represented at arrow 272 leading to block 274 in FIG. 12D, the monitor issues a visual and/or audible cue to the operator to start the injection of the indicator (e.g., ICG dye) at TI₁ seconds before the specified end of the Valsalva maneuver. The practitioner may be provided with a visual cue via, for example, an illuminated LED light affixed on or near the flow sensor 44 as seen in FIG. 1, so that the cue may be conveyed without difficulty. By way of example, if the selected Time Interval, TI₁ is 2.60 seconds and the programmed Valsalva maneuver duration is 5.00 seconds, then said visual and/or audible cue to the operator is issued 5.00 seconds less 2.60 seconds, which equals 2.4 seconds after the detected start of the Valsalva maneuver at block 266.

Still referring to FIG. 12D, the flow sensor 44 seen in FIG. 1 will detect the flow of indicator. As seen at arrow to query block 278, the flow sensor will continue to check for the detection of the start of the flow of the indicator. When said flow of indicator is detected, as represented by arrow 282 to block 284, time clock t₁ is set to zero at the moment the flow sensor detects the start of the injection of indicator and the countdown (i.e., count up) to the specified Time Interval, TI₁ begins. Then, as represented by arrow 286 to query block 288, monitor continues to check to determine if the elapsed time, t₁ now equals the selected Time Interval, TI₁.

Once the elapsed time, t₁ equals the selected Time Interval, TI₁ as seen at arrow 292, the program continues to block 294 at which time the solenoid 84, as seen in FIG. 1, is energized, forcing a rapid retraction of the piston 94 in the vacuum/pressurization subassembly 87 and inducing a vacuum level (i.e., negative pressure level) sufficient to rapidly retract the shuttle in the mouthpiece assembly 20. Upon the retraction of the shuttle, as seen in FIG. 4, one or more vents 26 in the mouthpiece assembly are in low-resistance, air-flow communication with the mouth 4 and lungs of patient 8 as seen in FIGS. 1 and 4, thereby inducing the immediate expiration of air from the lungs of patient 8 and the corresponding end of the Valsalva maneuver. By this process, the Valsalva maneuver is controllably ended at a precise, predetermined time interval after the detected start of indicator injection and does not depend on the response time of patient 8 to any audible and/or visual cues to initiate their own action to end the Valsalva maneuver.

Still referring to block 294 of FIG. 12D as well as FIG. 1, within a predetermined time after solenoid 84 is energized and vents 26 “open” (e.g., a time period of about 5.0 seconds), the solenoid is de-energized at which time the restraining force holding piston 94 in the retracted position, as seen in FIG. 1, becomes zero. At this moment, the force exerted by compression spring 120 in its contracted state induces the rapid return of piston 94 to its distal starting position. The rapid return of piston 94 to its distal starting position induces a positive pressure in the internal tubing assembly 100 and extension tubing 36, thereby forcing the shuttle in the mouthpiece assembly 20 to return to its proximal starting (“home”) position as seen in FIGS. 1 and 3.

Referring next to FIG. 12E, arrow 296 reappears, extending to block 298, which measures the peak amplitude, and for each of the channels N, calculates the peak amplitude signal, S_{NORMAL}(N) for normal indicator/dilution curves associated with indicator and blood flowing through a normal pathway in the lungs. Then, as represented at arrow 300 to block 302, a query is made as to whether the measured signal for at least one channel is equal to or greater than a minimum designated signal. Where it is not, then as represented at arrow 304 to block 306, the practitioner is alerted with an audible/visual error message that there is insufficient coupling between the sensor and blood-born indicator in the tissue, and the test is ended.

Where that signal is greater than the minimum required signal, then as represented at arrow 308 and block 310, the peak amplitude signal for each channel with a premature indicator/dilution curve prior to the normal indicator/dilution curve, or the peak amplitude of an inflection in the up-slope portion of the start of the normal curve (both being associated with a right-to-left shunt), are measured. Where a non-zero premature indicator/dilution curve/inflection signal result is occurring, then as represented at arrow 312 to block 314, the conductance associated with a right-to-left cardiac shunt is calculated. This can be done using a ratio obtained by dividing the shunt curve/inflection signal peak amplitude by the normal curve signal peak amplitude, for each pair of normal curve peak amplitudes and shunt signal peak amplitudes existing for each channel. The maximum ratio of the shunt signal peak amplitude over its corresponding normal curve peak amplitude is displayed as the shunt conductance index.

Next, as represented at arrow 316 to block 318, an inquiry is made to whether the procedure count index, PFLAG, is now equal to 2. Where PFLAG is equal to 2, then as represented at arrow 328 to symbol 330, the test is ended. Where the procedure count index, PFLAG is not equal to 2, then as represented at arrow 320, the procedure count index, PFLAG is set equal to 2 and the second Time Interval, TI₂ is selected. In this regard, it should be noted that this exemplary embodiment of the invention assumes that a complete test for the presence of a right-to-left cardiac shunt requires that two tests be performed with two different Time Intervals, TIₙ (e.g., 2.60 and 1.60 seconds). For this embodiment, the program then continues as represented at arrow 324 to Node A at 326. The program is now prepared to proceed to the second of two right-to-left shunt test procedures, which begins at block 242 and ends at block 330. In this regard, Node A 326 reappears in FIG. 12C in conjunction with arrow 240 extending to arrow 236.

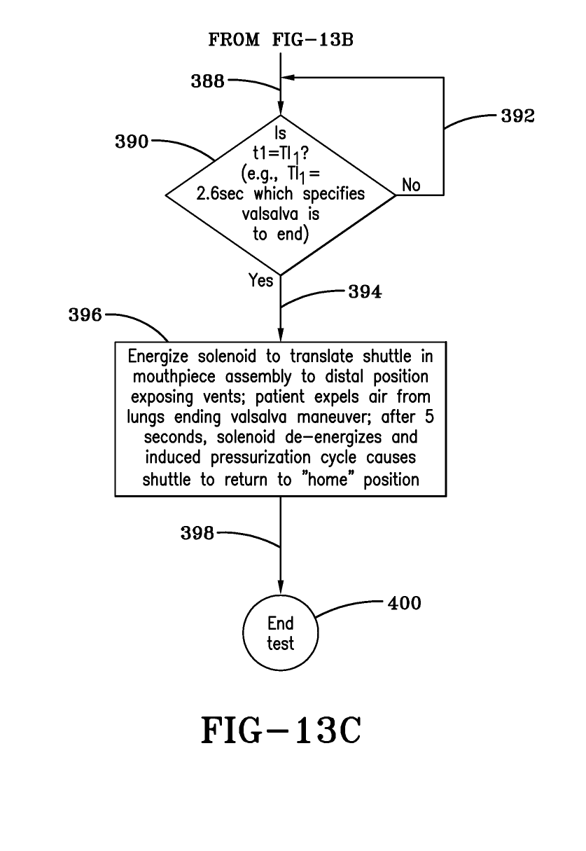

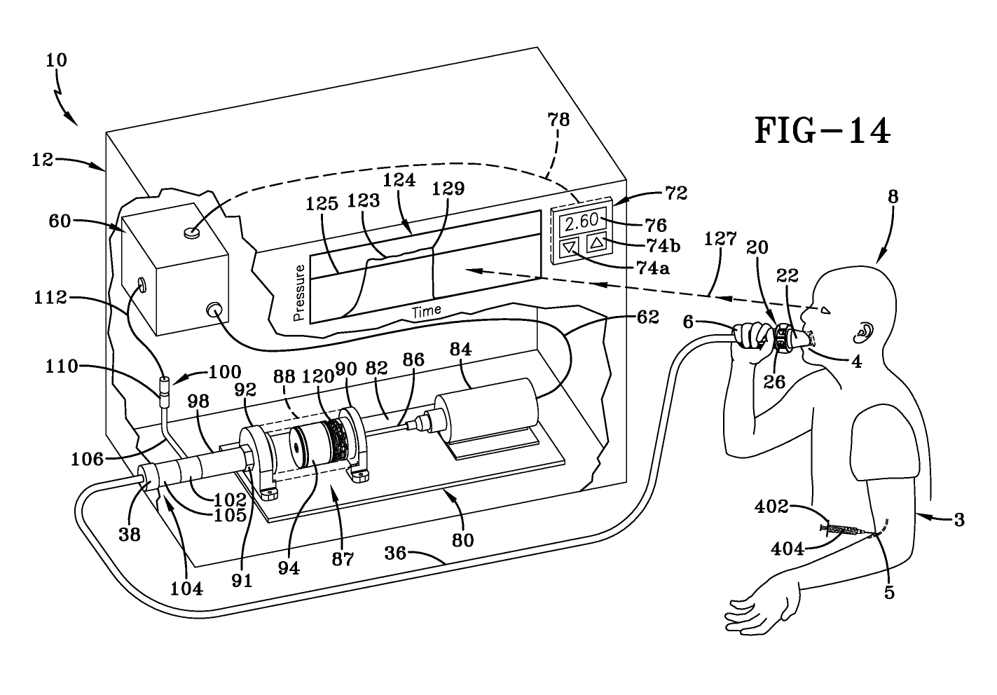

A general flow chart of the operation of another exemplary embodiment of a system of the invention is described in FIGS. 13A through 13C. These figures are combined as labeled thereon to provide a flow chart describing the system and method for the performance of a Valsalva maneuver. By way of example, this flow chart of the operation of an embodiment of the invention corresponds to the performance of a Valsalva maneuver required for the detection of a right-to-left cardiac shunt using the Transcranial Doppler (TCD) method, Transthoracic Echocardiography (TTE) method, as well as other methods using indicator dyes to detect the presence of a right-to-left cardiac shunt. The apparatus, system and method used for the performance of a Valsalva maneuver according to this embodiment, are seen in FIGS. 2 through 11, 13A through 13C and 14.

Beginning as represented by symbol 340 and continuing as represented by arrow 342 to block 344, the monitor 10 carries out system initialization with the establishment of default parameters. Time Interval, TI is selected and the elapsed time t₁ is set to zero. At this step, mouthpiece assembly 20 is connected to monitor 10 using quick-disconnect fitment 50 at distal end of extension tubing 36 as seen in FIG. 14. The program continues, as represented at arrow 346 and block 348, to provide for placement of mouthpiece assembly in the hand of patient 8 and to instruct patient 8 to place the ergonomic tube 22 in his or her mouth 4 of as seen at 20 in FIG. 14. Still referring to FIG. 13A, from block 348, arrow 350 leads to the “Start of Test” indication at block 352.

Arrow 354 reappears in FIG. 13B extending to block 356, to provide for the initialization of the shuttle location in the mouthpiece assembly 20 by activating one evacuation cycle followed by one pressurization cycle to position shuttle in the initial “home” position within the tubular body of the mouthpiece assembly 20. Next, the program starts measurement as represented at arrow 358 extending to block 360, wherein instructions are provided to the patient to begin the Valsalva maneuver by exhaling into the ergonomic tube 22 of the mouthpiece assembly 20, as seen in FIG. 14, to reach and maintain the target pressure level until the monitor terminates the Valsalva maneuver automatically after the specified Time Interval, TI₁ has elapsed.

Generally, the Valsalva maneuver procedure is accompanied by some form of display on monitor 10. Turning momentarily to FIG. 14, a line graph 123 is provided along with a minimum exhalation pressure level 125, represented as a solid line, giving the patient the actual real-time measurement of the pressure being exerted by the patient during the Valsalva maneuver. The graph display 124 shows exhalation pressure versus elapsed time. In FIG. 14, the patient's Valsalva exhalation pressure has just been released automatically by the combined operation of the solenoid-driven vacuum/pressurization assembly and mouthpiece assembly seen at release time point 129 in FIG. 14. As seen at display screen 124 of FIG. 14, the Valsalva maneuver was properly ended with the graph 123 displaying that the patient held the proper pressure (with some acceptable variation) during the duration of the Valsalva maneuver.

Returning to FIG. 13B, as represented at arrow 362 to block 364, the exhalation pressure created by the patient during the Valsalva maneuver is continuously measured and displayed on the monitor, as explained in connection with FIG. 14, and is compared to the ideal Valsalva curve or required minimum exhalation pressure level 125. As represented by arrow 366 to block 368, the exhalation pressure is queried and it is determined whether it falls within a measurable range, for example from 0-4000 analog-to-digital converted (ADC) units. If not, arrow 372 is followed to block 374, wherein a system fault is displayed and the test is ended. If the measured exhalation pressure is within an expected range, arrow 370 is followed to block 376.

Still referring to FIG. 13B, block 376 poses the query as to whether the exhalation pressure level is above or equal to the targeted pressure, for example, 35 mm Hg. In the event that it is not, as represented at arrow 378 and block 380, the operator is alerted with an audible alarm or visual error message to instruct the patient to increase pressure to meet or exceed the target exhalation pressure level. Where the exhalation pressure is appropriate, the program continues as represented at arrow 384.

As represented at arrow 384, extending from the query at block 376 and leading to block 386, the Time Interval elapsed time clock t₁ is set to t₁=0 and begins the countdown (i.e., count up) to the specified Time Interval value, TI₁. By way of example, at a point in time appropriate to the right-to-left cardiac shunt detection method being used and corresponding to the selected Time Interval, TI₁ the operator injects indicator 404 using syringe 402 into the antecubital vein 5 at arm 3 of patient 8 as seen in FIG. 14. Said indicator 404 in this example may be a contrast agent containing a multiplicity of small air bubbles for detection using ultrasound-based methods (see above-listed References 5 through 8) or a dye detectable by a spectrophotometric method such as pulsed dye densitometry (see above-listed Reference 15).

Arrow 388 reappears in FIG. 13C extending to query block 390. Then, as represented by query block 390, monitor continues to check to determine if the elapsed time, t₁ now equals the selected Time Interval, TI₁. Once the elapsed time, t₁ equals the selected Time Interval, TI₁ as seen at arrow 394, the program continues to block 396 at which time the solenoid 84, as seen in FIG. 14, is energized by power supply (not shown) within controller 60, forcing a rapid retraction of the piston 94 in the vacuum/pressurization subassembly and inducing a vacuum level (i.e., negative pressure level) sufficient to rapidly retract the shuttle in the mouthpiece assembly 20.

Upon the retraction of the shuttle, as seen in FIG. 4, one or more vents 26 in the mouthpiece assembly 20 are in low-resistance, air-flow communication with the mouth 4 and lungs of patient 8 as seen in FIGS. 4 and 14, thereby inducing the immediate expiration of air from the lungs of patient 8 and the corresponding end of the Valsalva maneuver. By this process, the Valsalva maneuver is controllably ended at a precise, predetermined time interval after the detected start of indicator injection and does not depend on the response time of patient 8 to any audible and/or visual cues to initiate their own action to end the Valsalva maneuver.

Still referring to block 396 of FIG. 13C as well as FIG. 14, within a predetermined time after solenoid 84 is energized and vents 26 “open” (e.g., a time period of 5.0 seconds), solenoid 84 is de-energized at which time the restraining force holding piston 94 in the retracted position, as seen in FIG. 14, becomes zero. At this moment, the force exerted by compression spring 120 in its contracted state induces a rapid return of piston 94 to its distal starting position. The rapid return of piston 94 to its distal starting position induces a positive pressure in the internal tubing assembly 100 and extension tubing 36, thereby forcing the shuttle in the mouthpiece assembly 20 to return to its proximal starting (“home”) position as seen in FIGS. 3 and 14. Finally, as represented at arrow 398 to symbol 400, the test is ended.

The present application, by way of U.S. Provisional Application No. 61/696,409 to which it claims benefit, herein incorporates by reference the subject matter of U.S. patent application Ser. No. 12/754,888 filed Apr. 6, 2010 and Ser. No. 12/418,866 filed Apr. 6, 2009; U.S. Provisional Application Nos. 61/156,723 filed Mar. 2, 2009 and 61/080,724 filed Jul. 15, 2008; and PCT applications PCT/US09/50630 filed Jul. 15, 2009 and PCT/US11/31433 filed Apr. 6, 2011. All citations referred to therein are also expressly incorporated herein by reference.

All terms not specifically defined herein are considered to be defined according to Dorland's Medical Dictionary, and if not defined therein according to Webster's New Twentieth Century Dictionary Unabridged, Second Edition.

While certain exemplary embodiments of the invention are described in detail above, the scope of the invention is not to be considered limited by such disclosure, and modifications are possible without departing from the spirit of the invention as evidenced by the following claims:

What is claimed is:

1. A Valsalva maneuver control device, comprising:
a mouthpiece assembly having a first end for insertion into the mouth of a patient and a second end for connection to a vacuum/pressurization assembly;
a solenoid-driven vacuum/pressurization assembly connected, by tubing, to the second end of the mouthpiece assembly;
a controller for operating the solenoid-driven vacuum/pressurization assembly to either provide pressure resistance against air exhaled into the mouthpiece assembly by the patient or to allow said air to pass therethrough, the controller including a timer; and
a monitor having a display for guiding the patient through performance of the Valsalva maneuver;
wherein the controller is operative to actuate the vacuum/pressurization assembly to rapidly discontinue the pressure resistance against air exhaled into the mouthpiece assembly by the patient as directed by the timer so as to end the Valsalva maneuver at a desired time.

2. The Valsalva maneuver control device of claim 1, further comprising a catheter set and dye syringe for injecting an indicator dye into the blood stream of patient at a peripheral location, a flush syringe in communication with the catheter set for introducing a second injection of isotonic saline, and a flow sensor for detecting the passage of indicator dye, the flow sensor in electronic communication with the controller.

3. The Valsalva maneuver control device of claim 2, wherein the controller is programmed to initiate the Valsalva maneuver upon detection of dye injection.

4. The Valsalva maneuver control device of claim 2, wherein the controller is programmed to actuate the vacuum/pressurization assembly to end the Valsalva maneuver at a particular elapsed time after the detection of dye injection.

5. The Valsalva maneuver control device of claim 2, wherein the elapsed time is equal to a specified time interval of 1.60 or 2.60 seconds.

6. The Valsalva maneuver control device of claim 1, wherein the mouthpiece assembly includes a plurality of vents, and a reciprocating shuttle that is actuatable by the solenoid-driven vacuum/pressurization assembly to selectively cover or uncover the vents so as to either provide pressure resistance against air exhaled into the mouthpiece assembly by the patient or to allow said air to pass therethrough.

7. The Valsalva maneuver control device of claim 6, wherein activation of the solenoid-driven vacuum/pressurization assembly will produce a vacuum that will displace the shuttle in a manner that uncovers the mouthpiece vent holes, thereby removing the pressure resistance against air exhaled into the mouthpiece assembly so as to cause a rapid exhalation by the patient that will end the Valsalva maneuver.

8. The Valsalva maneuver control device of claim 1, wherein the monitor display graphically indicates whether the patient is creating a sufficient level of exhalation pressure while performing the Valsalva maneuver and issues a warning if the created pressure drops below a threshold pressure level.

9. The Valsalva maneuver control device of claim 1, wherein the controller and monitor are part of a single unit.

10. A mouthpiece for use in performing a Valsalva maneuver, comprising:
a mouthpiece blow tube for insertion into the mouth of a patient, and in fluid connection with a tubular body;
vent holes located in the tubular body;
a movable shuttle located in the tubular body, the shuttle alternately reciprocatable between a first position that isolates the vent holes, and a second position that exposes the vent holes; and
an extension tube providing fluid communication between the tubular body and a vacuum/pressurization device;
wherein, the shuttle is adapted such that the application of vacuum to the tubular body will cause the shuttle to move to the second position, thereby exposing the vent holes and rapidly de-pressurizing the mouthpiece.

11. The mouthpiece of claim 10, wherein the tubular body includes baffle plates that are designed and located to direct air exhaled by a patient into the mouthpiece away from the face of the patient when the vents are exposed and air is allowed to be rapidly expelled from the patient's lungs.

12. The mouthpiece of claim 10, wherein the shuttle is adapted to remain in its current position when a negative pressure or positive pressure is initially applied to the tubular body, but to move to the alternative position when the negative pressure or positive pressure is reversed.

13. The mouthpiece assembly of claim 10, further comprising a pair of O-rings in contact with inner walls of the tubular body to further enable the movement of the shuttle upon application of a negative pressure or positive pressure to the tubular body.

14. The mouthpiece assembly of claim 13, wherein a biocompatible lubricant is used in combination with the O-rings.

15. The mouthpiece assembly of claim 10, further comprising a small diameter hole in the tubular body, the hole providing a sufficiently large flow factor so as to enable pressure equalization and dynamic exhalation pressure measurement, and simultaneously a sufficiently small flow factor to enable negative pressures or positive pressures rapidly created within the tubular body to induce rapid movement of the shuttle from a vents closed position during a Valsalva maneuver to a vents open position at the moment of an intended Valsalva maneuver release.

16. A Valsalva maneuver control method, comprising:
providing a mouthpiece assembly having a first end for insertion into the mouth of a patient and a second end for connection to a vacuum/pressurization assembly;
providing a solenoid-driven vacuum/pressurization assembly connected, by tubing, to the second end of the mouthpiece assembly;
providing a controller for operating the solenoid-driven vacuum/pressurization assembly to either provide pressure resistance against air exhaled into the mouthpiece assembly by the patient or to allow said air to pass therethrough, the controller including a timer;
providing a monitor having a display for guiding the patient through performance of the Valsalva maneuver;
initiating the Valsalva maneuver;
using the timer of the controller to determine when to terminate the Valsalva maneuver;
at the determined time of Valsalva maneuver termination, using the controller to actuate the vacuum/pressurization assembly to create a vacuum that rapidly discontinues the pressure resistance against air exhaled into the mouthpiece assembly by the patient, thereby causing a rapid exhalation by the patient and release of the Valsalva maneuver at the desired time.

17. The method of claim 16, further comprising:
providing a catheter set and dye syringe for injecting an indicator dye into the blood stream of patient at a peripheral location, a flush syringe in communication with the catheter set for introducing a second injection of isotonic saline, and a flow sensor for detecting the passage of indicator dye, the flow sensor in electronic communication with the controller;
injecting dye and, subsequently saline, into the patient via the catheter set; and
using the Valsalva maneuver control method in conjunction with the dye injection for the purpose of detecting a cardiac shunt in the patient.

18. The method of claim 17, wherein the controller initiates the Valsalva maneuver upon detection of dye injection and operates to end the Valsalva maneuver at 1.60 or 2.60 seconds after dye injection.

19. The method of claim 16, further comprising providing on the monitor display a graphical indication as to whether the patient is creating a sufficient level of exhalation pressure while performing the Valsalva maneuver, and issuing a warning if the created pressure drops below a threshold pressure level.

20. The method of claim 16, further comprising providing a controlled leak in the mouthpiece assembly that forces a patient to create the required Valsalva maneuver pressure using their diaphragm and not their cheek muscles.
